(19) United States
(12) Patent Application Publication
(10) Pub. No.: US 2013/0143969 A1
(43) Pub. Date: Jun. 6, 2013

(54) CURCUMIN COMPOSITIONS AND USES THEREOF

(71) Applicants: Zhongfa Liu, Columbus, OH (US); Ming Heung Chiu, Columbus, OH (US); Wei Chen, Columbus, OH (US)

(72) Inventors: Zhongfa Liu, Columbus, OH (US); Ming Heung Chiu, Columbus, OH (US); Wei Chen, Columbus, OH (US)

(73) Assignee: THE OHIO STATE UNIVERSITY, Columbus, OH (US)

(21) Appl. No.: 13/817,260

(22) PCT Filed: Aug. 17, 2011
(86) PCT No.: PCT/US11/48090

§ 371 Date: Feb. 15, 2013

Related U.S. Application Data
(60) Provisional application No. 61/374,480, filed on Aug. 17, 2010.

(51) Int. Cl. A61K 31/12

(52) U.S. Cl. CPC A61K 31/12
USPC 514/679

(57) ABSTRACT

A composition and method of increasing the bioavailability of curcumin is provided. A synergistic combination of excipient polymers provides increased bioavailability thereby increasing the plasma concentration of curcumin and its metabolite curcumin O-glucuronide. The curcumin pharmaceutical compositions are suitable for modifying DNA methylation and treating diseases such as cancer.

### CROSS-REFERENCE TO RELATED APPLICATIONS

This application claims the benefit of and priority to prior filed co-pending Provisional Application Ser. No. 61/374,480, filed Aug. 17, 2010, which is expressly incorporated herein by reference in its entirety.

### FIELD OF THE INVENTION

The invention relates to pharmaceutical or nutraceutical compositions and its anti-cancer activity. More particularly, the invention relates to gel compositions of curcumin and its hypomethylation activity.

### BACKGROUND OF THE INVENTION

Various physiological factors reduce the availability of drugs prior to their entry into the systemic circulation. For example, whether a drug is taken with or without food may affect absorption, other drugs taken concurrently may alter absorption and first-pass metabolism, and intestinal motility may alter the dissolution of the drug and may affect the degree of chemical degradation of the drug by intestinal microflora. Disease states affecting liver metabolism or gastrointestinal function may also have an effect. Other factors may include, but are not limited to, physical properties of the drug, e.g., hydrophobicity, pKa, solubility, and chemical stability. As such, bioavailability of orally administered chemicals is difficult, if not impossible, to predict.

Curcumin, a naturally-occurring and bioactive component in Curcuma longa, has been shown various biological activities and pharmacological actions, such as anti-tumor, anti-inflammatory, anti-virus, anti-oxidation and anti-HIV in preclinical settings and to be well tolerated up to 12 g/day for 3-4 months in clinical settings. However, the pharmacokinetic (PK) studies of curcumin in rodents and humans have consistently reported poor systemic bioavailability.

A number of factors could reduce the oral bioavailability of curcumin. These factors include: 1) rapid glucuronidation/sulfonation of curcumin's phenolic hydroxyl groups; 2) its high first pass clearance; 3) its instability in aqueous solution at pH equal to and above 7; and 4) its hydrophobic property; and 5) its low water solubility at acidic pH. Thus, when curcumin is administered as a dry powder, most of the curcumin is never absorbed and is excreted unchanged. Thus, solubilization is critical to increasing its oral bioavailability.

In rodents, poor absorption has been observed for oral administration. For example, no curcumin was detected in the urine and dose-independent low curcumin plasma level (5 nM) and high curcumin levels (>3 μmol/g) was found in the feces after dietary feeding of curcumin (0.1%˜0.5%) to rats. The elimination half-life (T_{1/2}) of orally administered curcumin was reported to be 1.7±0.5 hr in rats. After oral administration to mice of curcumin solution or fortified food up to the dose of 1 g/kg, the maximal plasma level was 0.6 μM.

Similar to rodent studies, the oral bioavailability of curcumin is also poor in humans. For example, in 25 patients with precancerous lesions, the mean plasma levels were 0.19, 0.20, and 0.60 μg/ml (0.51, 0.54, and 1.6 μM) after taking 4, 6 and 8 g per day for 3 months, respectively. (Brenner, D. E., et al., Cancer Epidemiology, Biomarkers & Prevention Jun. 2008 17; 1411.) In an effort to improve the bioavailability of curcumin, several methods including adjuvant, phospholipid complexes, nano-particle formulation, micro emulsion, and novel curcumin analogs have been evaluated with marginal success. As such, the limited oral bioavailability of curcumin in humans poses a major clinical obstacle to its therapeutic use since achieving adequate plasma levels may be essential for its desirable pharmacological effects.

Moreover, many cancers are associated with abnormal DNA methylation profiles. DNA methylation of cytosine bases in the context of the sequence 5′-cytosine-phospho-guanosine (CpG) in gene promoter regions is an epigenetic mechanism that controls gene transcription, genome stability and genetic imprinting in collaboration with post-translational histone modification, including acetylation and methylation. This process is concertedly mediated by DNA methyltransferases (DNMT1, DNMT3a, and DNMT3b) in the presence of s-adenosyl-methionine (SAM) that serves as a methyl donor for methylation of cytosine. Aberrant hypermethylation of CpG rich regions (>55% CG content, the so-called CpG islands) in the promoter of Tumor Suppressor Genes (TSGs) results in their transcriptional silencing in a variety of solid and blood cancers. In-vitro and in-vivo treatment with DNA methylation inhibitors has proven to be effective in restoring the expression of TSGs involved in cell cycle regulation (e.g. p15^{INK4B}), proliferation, apoptosis, and differentiation. Decitabine and 5-azacitidine are two Food and Drug Administration (FDA) approved azanucleosides for treatment of Myelodysplastic Syndrome (MDS). However, their low specificity, rapid onset of chemo-resistance, and toxicities (i.e., myelosuppression), restriction to S phase specificity pose significant limitations for their use, especially for hypoproliferative cancer cells including cancer stem cells. Thus, discovery and development of novel DNA methylation inhibitors that are more effective and less toxic are essential. Several non-nucleoside DNA methylation inhibitors, such as procainamide and RG108, have been identified. However, none of them show comparable hypomethylating activity to that of azanucleosides.

Curcumin has been used for the treatment of inflammation, skin wounds, cough, as well as certain tumors. The world-wide consumption of curcumin has led to extensive studies aiming at elucidating molecular mechanisms for its anti-cancer and biological activities, such as being a free radical scavenger, inhibition of NF-KB translocation, and induction of glutathione S-transferase. Recently, curcumin has been found to modulate histone acetylation as a new member of histone deacetylase and histone acetyl transferase inhibitors in Raji cells and NB4 cells, respectively. Curcumin also alters the expression of TSGs, such as E-cadherin-11 in MDA-MB-231 cells, following a low concentration long-term treatment. Notably, curcumin and its analogs have been shown to have potent inhibitory activity on M. SssI, an DNMT1 analog from bacterial, with an IC₅₀ of 30 nM, and to induce significant global DNA hypomethylation in leukemia cells at its in-vivo achievable concentrations. All together, curcumin has a great potential to be used as an effective DNA methylation inhibitor, however, as mentioned above the potential therapeutic benefits of curcumin are limited by its very limited bioavailability. Therefore, new compositions and methods are needed to increase the bioavailability of curcumin in subjects.

### SUMMARY OF THE INVENTION

The present invention is premised on the realization that bioavailability of curcumin can be increased by utilizing an appropriate gel formulation. More particularly, the present invention is premised on the realization that a highly concentrated gel formulation of curcumin in at least two excipient polymers may be employed to increase the bioavailability of curcumin When orally administered to a subject, the highly concentrated gel formulation of curcumin provides previously unattainable curcumin plasma concentrations.

In accordance with an embodiment of the invention, a pharmaceutical composition is provided that comprises curcumin and at least two excipient polymers selected from the group consisting of a polyethoxylated castor oil, a polyoxyethylene sorbitan ester, and a polyethylene glycol (PEG).

According to another embodiment of the invention, a method of making an oral gel pharmaceutical composition is provided that comprises forming a curcumin suspension in a liquid comprising a first excipient polymer selected from the group consisting of a polyethoxylated castor oil, a polyoxyethylene sorbitan ester, and a polyethylene glycol (PEG); heating the curcumin suspension to a temperature between a range from about 30° C. to about 150° C. to form a homogenous curcumin gel; and diluting the homogenous curcumin gel with a liquid comprising a second excipient polymer selected from the group consisting of a polyethoxylated castor oil, a polyoxyethylene sorbitan ester, and a polyethylene glycol (PEG).

According to yet another embodiment of the present invention, a method of increasing a bioavailability of curcumin is provided that comprises administering an oral curcumin gel pharmaceutical composition to a subject, wherein the oral curcumin gel comprises curcumin, and at least two excipient polymers selected from the group consisting of a polyethoxylated castor oil, a polyoxyethylene sorbitan ester, and a polyethylene glycol (PEG).

According to yet another embodiment of the present invention, a method of modulating DNA methylation and/or inhibiting DNA methylation is provided using a pharmaceutical composition that comprises curcumin and at least two excipient polymers selected from the group consisting of a polyethoxylated castor oil, a polyoxyethylene sorbitan easter, and a polyethylene glycol (PEG).

According to yet another embodiment, a method of treating cancer, such as leukemia and/or breast cancer, is provided using a pharmaceutical composition that comprises curcumin and at least two excipient polymers selected from the group consisting of a polyethoxylated castor oil, a polyoxyethylene sorbitan ester, and a polyethylene glycol (PEG).

### DESCRIPTIONS OF THE DRAWINGS

FIG. 1A is mass spectra of curcumin;

FIG. 1B is a tandem mass spectra of curcumin;

FIG. 1C is mass spectra of tetrahydrocurcumin (THC);

FIG. 1D is a tandem mass spectra of THC;

FIG. 1E is mass spectra of OSU-Arg;

FIG. 1F is a tandem mass spectra of OSU-Arg;

FIG. 2A is a chromatogram of blank mouse plasma.

FIG. 2B is a chromatogram of curcumin extracted from spiked mouse plasma.

FIG. 2C is a chromatogram of blank mouse plasma.

FIG. 2D is a chromatogram of THC extracted from spiked mouse plasma.

FIG. 2E is a chromatogram of blank mouse plasma.

FIG. 2F is a chromatogram of OSU-Arg extracted from spiked mouse plasma.

FIG. 3 is a graph illustrating the plasma concentration after oral administration of curcumin formulations according to embodiments of the invention.

FIG. 4A is a graph illustrating the plasma concentration after oral administration of curcumin formulations according to embodiments of the invention.

FIG. 4B is a graph illustrating the plasma concentration after oral administration of curcumin formulations according to embodiments of the invention.

FIG. 4C is a graph illustrating the plasma concentration after oral administration of curcumin formulations according to embodiments of the invention.

FIG. 5 is a plasma profile comparing the plasma concentration after oral administration of a curcumin formulation according to embodiments of the invention with a suspension of curcumin.

FIG. 6A is a representative LC/MS/MS chromatogram of a plasma extract after oral administration of a curcumin formulation according to embodiments of the invention.

FIG. 6B is a representative LC/MS/MS chromatogram of a plasma extract after oral administration of a curcumin formulation according to embodiments of the invention.

FIG. 6C is a representative LC/MS/MS chromatogram of a plasma extract after oral administration of a curcumin formulation according to embodiments of the invention.

FIG. 6D is a representative LC/MS/MS chromatogram of a plasma extract after oral administration of a curcumin formulation according to embodiments of the invention.

FIG. 6E is a representative LC/MS/MS chromatogram of a plasma extract after oral administration of a curcumin formulation according to embodiments of the invention.

FIG. 6F is a representative LC/MS/MS chromatogram of a plasma extract after oral administration of a curcumin formulation according to embodiments of the invention.

FIG. 7 is a graph illustrating the plasma profiles of curcumin metabolites after oral administration of a curcumin formulation according to embodiments of the invention.

FIG. 8 is a graph illustrating the stability of curcumin formulations according to embodiments of the invention.

FIG. 9 is a graph illustrating the time profile of the plasma level of curcumin after oral administration of a curcumin formulation according to embodiments of the invention.

FIG. 10A is a graph illustrating the antitumor growth activity of a curcumin formulation according to embodiments of the invention.

FIG. 10B is a graph illustrating the antitumor growth activity of a curcumin formulation according to embodiments of the invention.

FIG. 11A is a series of graphs illustrating the down regulation of DNMT1, DNMT3a, and DNMT3b mRNA levels by a curcumin formulation according to embodiments of the invention.

FIG. 11B is a series of representative protein blots illustrating the down regulation of DNMT1, DNMT3a, and DNMT3b protein levels by a curcumin formulation according to embodiments of the invention.

FIG. 11C is a graph illustrating the down regulation of DNMT1, DNMT3a, and DNMT3b mRNA levels by a curcumin formulation according to embodiments of the invention.

FIG. 11D is a series of representative protein blots illustrating the down regulation of DNMT1 protein levels by a curcumin formulation according to embodiments of the invention.

FIG. 11E is a graph illustrating the down regulation of DNMT1 mRNA levels by a curcumin formulation according to embodiments of the invention.

FIG. 11F is a series of representative protein blots illustrating the down regulation of DNMT1 protein levels by a curcumin formulation according to embodiments of the invention.

FIG. 12A is a graph illustrating the down regulation of p15 mRNA levels by a curcumin formulation according to embodiments of the invention.

FIG. 12B is a graph illustrating the down regulation of p15 mRNA levels by a curcumin formulation according to embodiments of the invention.

FIG. 12C is a graph illustrating the DNA methylation of the p15 promoter after exposure to a curcumin formulation according to embodiments of the invention.

FIG. 12D is a graph illustrating the DNA methylation of the p15 promoter after exposure to a curcumin formulation according to embodiments of the invention.

FIG. 13A is a graph illustrating the cell cycle distribution of cells treated with a curcumin formulation according to embodiments of the invention.

FIG. 13B is a graph illustrating cell viability after exposure to a curcumin formulation according to embodiments of the invention.

FIG. 14A is a graph illustrating the effect of a curcumin formulation according to embodiments of the invention on tumor size in mice.

FIG. 14B is a graph illustrating the effect of a curcumin formulation according to embodiments of the invention on tumor weight in mice.

FIG. 14C is a graph illustrating the effect of a curcumin formulation according to embodiments of the invention on body weight in mice with a tumor.

FIG. 15A is a graph illustrating the plasma concentration profile of curcumin in a human after administration of a curcumin formulation according to embodiments of the invention.

FIG. 15B is a graph illustrating the plasma concentration profile of curcumin-O-glucoronide in a human after administration of a curcumin formulation according to embodiments of the invention.

FIG. 15C is a graph illustrating the plasma concentration profile of curcumin in a human after administration of a curcumin formulation according to embodiments of the invention.

### DETAILED DESCRIPTION OF THE INVENTION

In accordance with embodiments of the present invention, a composition comprising curcumin is provided that unexpectedly provides increased bioavailability beyond that achieved by prior formulations. According to embodiments of the present invention, the curcumin composition comprises at least two excipient polymers selected from the group consisting of a polyethoxylated castor oil, a polyoxyethylene sorbitan ester, and a polyethylene glycol (PEG).

In accordance with the present invention, curcumin is the primary active ingredient of the pharmaceutical composition. Curcumin is the principal curcuminoid of the popular Indian spice turmeric, which is a member of the ginger family (Zingiberaceae). Natural curcumin may also contain small quantities of desmethoxycurcumin and bisdesmethoxycurcumin Curcumin can exist in at least two tautomeric forms, diketone and ketone-enol, which are shown below.

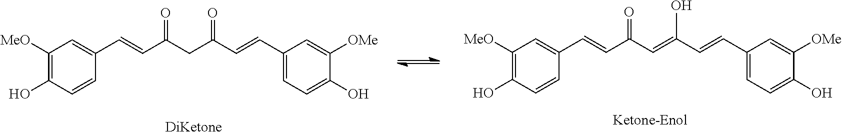 [SMILES: COc1cc(/C=C/C(=O)CC(=O)/C=C/c2ccc(O)c(OC)c2)ccc1O ; COc1cc(/C=C/C(=O)/C=C(O)/C=C/c2ccc(O)c(OC)c2)ccc1O]

The curcumin utilized in various embodiments of the present invention is not particularly limited to any particular form or supplier and includes curcumin derived from sources other than turmeric such as from other plants as well as synthetic curcumin. An exemplary curcumin that may be used is commercially-available from Acros Organics (product number #218580100).

In addition to the active ingredient curcumin, pharmaceutical compositions in accordance with embodiments of the present invention further comprises pharmaceutically acceptable carriers comprising one or more buffers, excipients, salts, preservative, auxiliaries and the like which facilitate processing of the active compounds into preparations which may be used pharmaceutically. The preparations are particularly formulated for oral administration. Appropriate pharmaceutically acceptable carriers are known to those of ordinary skill in the art and may be found in, for example, Remington: The Science and Practice of Pharmacy (20th Ed.) Lippincott, Williams & Wilkins (2000).

Examples of excipients include, but are not limited to, polyoxyethylene sorbitan esters (known as polysorbate or TWEEN®), polyethoxylated castor oil (Cremophor® EL), polyethylene glycols (PEG), poloxamer, methyl glucose sesquistearate, PEG-20 methyl glucoside sesquistearate, Steareth-21, polyethylene glycol 20 sorbitan monostearate, polyethylene glycol 60 sorbitan monostearate, polyethylene glycol 80 sorbitan monostearate, Steareth-20, Ceteth-20, PEG-100 stearate, sodium stearoyl sarcosinate, hydrogenated lecithin, sodium cocoylglyceryl sulfate, sodium stearyl sulfate, sodium stearoyl lactylate, PEG-20 glyceryl monostearate, sucrose monostearate, sucrose polystearates, polyglyceryl 10 stearate, polyglcyeryl 10 myristate, steareth 10, DEA oleth 3 phosphate, DEA oleth 10 phosphate, PPG-5 Ceteth 10 phosphate sodium salt, PPG-5 Ceteth 10 phosphate potassium salt, steareth-2, PEG-5 soya sterol oil, PEG-10 soya sterol oil, diethanolamine cetyl phosphate, sorbitan monostearate, diethylene glycol monostearate, glyceryl monostearate, and the like and mixtures thereof.

In particular, pharmaceutical compositions of the present invention include at least one excipient polymer selected from the group consisting of a polyethoxylated castor oil, a polyoxyethylene sorbitan ester, and a polyethylene glycol (PEG). According to embodiments of the present invention, the pharmaceutical compositions include at least two excipient polymers selected from the group consisting of a polyethoxylated castor oil, a polyoxyethylene sorbitan ester, and a polyethylene glycol (PEG). According to yet another embodiment of the present invention, the pharmaceutical compositions include at least three excipient polymers selected from the group consisting of a polyethoxylated castor oil, a polyoxyethylene sorbitan ester, and a polyethylene glycol (PEG).

In one embodiment, the polyethoxylated castor oil may be Cremophor® EL (BASF Corporation, and commercially-available from Sigma Aldrich, Inc., St. Louis, Mo.). In another embodiment, the polyoxyethylene sorbitan ester may be Tween® 20, Tween® 30, Tween® 40, Tween® 50, Tween® 60, Tween® 65, Tween® 70, Tween® 80, Tween® 85, or a combination thereof (ICI Americas, Inc., and commercially-available from Sigma Aldrich, Inc., St. Louis, Mo.). In yet another embodiment, the polyethylene glycol (PEG) may have an average molecular weight of about 200, about 300, about 400, about 500 or about 600. For example, suitable PEGs include PEG 200, PEG 300, PEG 400, PEG 500 and PEG 600, which are commercially-available from Sigma Aldrich, Inc., St. Louis, Mo. In one example, the polyethylene glycol may be PEG 200, PEG 300, PEG 400, PEG 500, PEG 600 or a combination thereof. In another example, the polyethylene glycol is PEG 600.

The pharmaceutical compositions may be prepared by dissolving solid curcumin in an excipient. In one embodiment, a sample of solid curcumin may be suspended in a volume of polyethylene glycol and the resulting mixture may be stirred at room temperature. In one embodiment, the mixture is stirred at an elevated temperature until a homogenous solution is obtained. For example, the mixture may be heated to a temperature within the range from about 30° C. to about 150° C.; from about 50° C. to about 120° C.; from about 60° C. to about 100° C.

A curcumin concentration in the homogenous excipient solutions may be within the range from about 40 to about 250 mg/mL. In one example, the curcumin concentration in a homogenous excipient solution is about 100 to about 250 mg/mL. For example, where polyethylene glycol is the excipient, the curcumin concentration may be affected depending on the average molecular weight of the PEG excipient and the mixing temperature, as shown in Table 1 below.

TABLE 1
The solubility of curcumin in different PEGs and temperatures

| PEG | 200 | 300 | 400 | 500 | 600 |
|---|---|---|---|---|---|
| Final Conc. @ RT (mg/mL) | NA* | NA | 42.7 | NA | 42.0 |
| Final Conc. 60° C. (mg/mL) | NA | NA | 131.4 | NA | 136.0 |
| Final Conc. 100° C. (mg/mL) | 193.1 | 252.3 | 195 | 218.9 | 241.8 |

*NA: Not available

Homogenous curcumin solutions, which are formed by dissolving curcumin in a liquid comprising a first excipient, such as polyethylene glycol, may be diluted with a liquid comprising a second excipient selected from the group consisting of a polyethoxylated castor oil, a polyoxyethylene sorbitan ester, and a polyethylene glycol (PEG). For example, in one embodiment, the second excipient is a polyethoxylated castor oil (Cremophor® EL). In another embodiment, the second excipient is a polyoxyethylene sorbitan ester (such as Tween® 20, Tween® 30, Tween® 40, Tween® 50, Tween® 60, Tween® 65, Tween® 70, Tween® 80, Tween® 85 or a combination thereof). The volume ratio of the first excipient to the second excipient may be from about 99:1 to about 1:99. In one exemplary embodiment, the ratio of the first excipient to the second excipient, such as PEG to Cremophor® EL, may be within the range from about 1:1 to about 4:1, for example about 4:3 or about 2:1. In another exemplary embodiment, the ratio of the first excipient to the second excipient, such as PEG to polyethoxyethylene sorbitan ester, may be within the range from about 1:1 to about 10:1.

The pharmaceutical composition described herein may further contain one or more suitable solvents. Examplary solvents include dimethylsulfoxide (DMSO), ethanol, sesame oil, acetone, and dimethylformamide (DMF).

According to embodiments of the present invention, the pharmaceutical composition may be suitable for use in various formulations, such as oral formulations, e.g., as capsules, soft capsules, liquid gels, pills and dropping pills; suppository formulations; topical formulations, e.g., creams, ointments, and gels; or injection formulations, as are commonly understood in the art.

Pharmaceutical compositions suitable for use in the present invention include compositions include curcumin in an amount effective to achieve the intended purpose in the subject. Determination of the effective amounts is well within the capability of those skilled in the art, especially in light of the detailed disclosure provided herein. For example, according to one embodiment, a method of treating a disease sensitive to curcumin is provided, wherein the disease is characterized by abnormally rapid proliferation of tissue involved in the disease which is mediated by or associated with abnormally increased levels of DNA methylation. The method comprises administering to a mammalian patient in need of such treatment a therapeutically-effective amount of the pharmaceutical composition, wherein the therapeutically-effective amount of the pharmaceutical composition is sufficient to modulate DNA methylation. According to another embodiment, the therapeutically-effective amount is sufficient to inhibit abnormally increased levels DNA methylation such as occurs in some diseased tissues. According to yet another embodiment, the subject may be a human or a domesticated animal.

According to another embodiment of the present invention, a method for treating cancer in a subject is provided that comprises administering to the subject a therapeutically-effective amount of the pharmaceutical composition. According to yet another embodiment, the subject may be a human or a domesticated animal. According to embodiments of the present invention, the cancer may be leukemia or breast cancer.

According to embodiments of the invention, curcumin may generally be administered in the inventive pharmaceutical compositions over a dose range from about 1.0 micromole/kg/day to about 0.5 millimole/kg/day, and in any event the dose is sufficient to treat a disease sensitive to curcumin such as a cancer or a disease which is mediated by or associated with abnormally increased levels of DNA methylation. Those skilled in the art can determine the appropriate level of dosing needed for each formulation. As discussed in greater detail below, the dosing may be affected by the route of administration used for the pharmaceutical compositions.

According to embodiments of the invention, the pharmaceutical formulations may be prepared as a capsule for oral administration to a subject. For example, an amount of pharmaceutical formulation according to the invention is prepared and transferred to a two ended capped capsule to yield a final product. In one exemplary embodiment, 1.50 g curcumin is weighed and suspended in 10 mL PEGx. The mixture is heated to 60° C. to yield a solution of 150 mg/mL curcumin PEGx solution. Then, 5 mL chromophor EL is added to the above solution which yields a 100 mg/mL curcumin solution. A 1 mL aliquot of this solution is transferred to a two-end capped capsule gel to yield the final pharmaceutical composition product.

### Example 1

Pharmaceutical formulations prepared according to embodiments of the invention improve bioavailability of curcumin. For these examples, curcumin (95%) was purchased from Acros Organics. Tetrahydrocurcumin (THC) was prepared according to a published procedure (Ohtsu, et al., J. Med. Chem., 2002, 45(23), 5037-5042). The internal standard OSU-Arg was obtained from National Cancer Institute (Bethesda, Md., USA). Analytical HPLC grade methanol, acetonitrile, ethyl acetate and formic acid were purchased from Fisher Scientific (Pittsburgh, Pa., USA). Heparin-treated and EDTA-treated mouse plasma were purchased from Harlan Bioproducts (Indianapolis, Ind., USA). The phosphate buffer saline (PBS, pH 7.4) was purchased from Sigma-Aldrich (St. Louis, Mo., USA). All chemicals and reagents were used as received. An E-pure water purification system (Barnstead, Dubuque, Iowa) was used to obtain HPLC grade water (>18 mΩ).

The LC-MS system used consisted of a Finnigan TSQ Quantum EMR Triple Quadrupole mass spectrometer (Thermo Fisher Scientific Corporation, San Jose, Calif.) coupled to a Shimadzu HPLC system (Shimadzu, Columbia, Md.), which was equipped with a CBM-20A system controller, an LC-20 AD pump, a SIL-20AC auto-sampler, CTO-20A column oven, DGU-20A5 degasser and FCV-11AL valve unit. The temperature of the auto-sampler was set at 4° C. during operation. All operations were controlled by Finnigan Xcalibur software (Home Page Version 1.4 SR1) on a Windows XP operating system.

Curcumin, THC, and the internal standard (I.S.) OSU-Arg were separated on a C₈ column (2.1×50 mm, 5 μm, Thermo Hypersil-Keystone, Bellefonte, Pa., USA) coupled with a C₈ basic guard column (2.1×10 mm, 2 μm, Thermo Hypersil-Keystone, Bellefonte, Pa., USA) at the flow rate of 0.20 mL/min with an isocratic eluent consisting of 50% mobile phase A (water/0.1% formic acid, MP-A) and 50% mobile phase B (CH₃CN/0.1% formic acid, MP-B).

The mass spectrometer was operated in the positive ESI mode with a collision gas (Argon) pressure of 1.5 psi, a typical electro-spray needle voltage of 4700 V, a sheath nitrogen gas flow of 25 (arbitrary unit) and a heated capillary temperature of 325° C. The samples and the internal standard were analyzed by the multiple reaction monitor (MRM) mode using ion transitions at a proper collision energy (E) as follows: curcumin m/z 369.0>m/z 177.0 (E=30%), m/z 373.0>m/z 137.0 (E=30%) and internal standard OSU-Arg m/z 446.0>m/z 429.0 (E=35%). The mass spectrometer was tuned to its optimal sensitivity by the direct infusion of curcumin and THC solution.

Chemical Structures of the Standards.

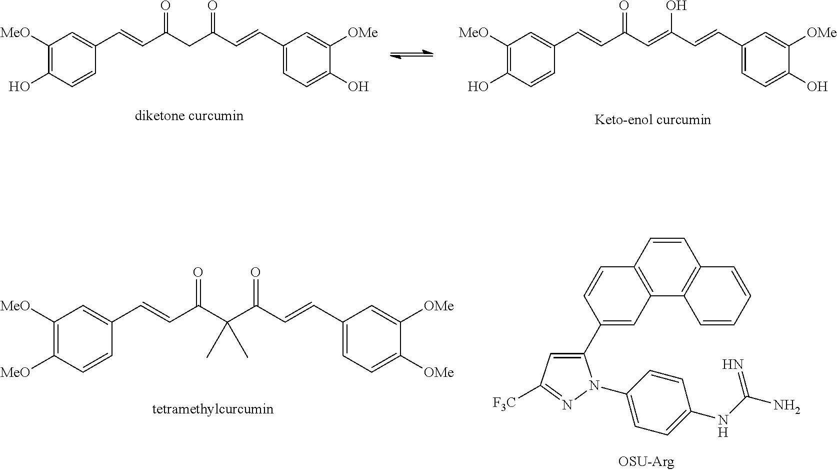 [SMILES: COc1cc(/C=C/C(=O)CC(=O)/C=C/c2ccc(O)c(OC)c2)ccc1O ; COc1cc(/C=C/C(=O)/C=C(O)/C=C/c2ccc(O)c(OC)c2)ccc1O ; COc1ccc(/C=C/C(=O)C(C)(C)C(=O)/C=C/c2ccc(OC)c(OC)c2)cc1OC ; N=C(N)Nc1ccc(-n2nc(C(F)(F)F)cc2-c2ccc3ccc4ccccc4c3c2)cc1]

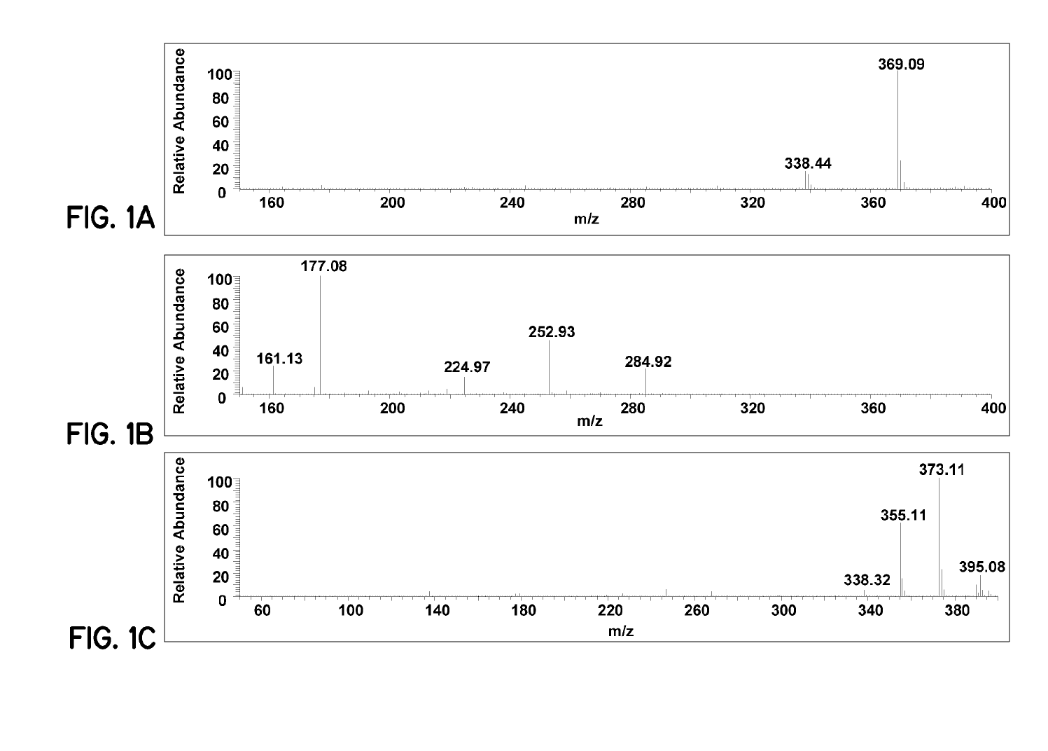

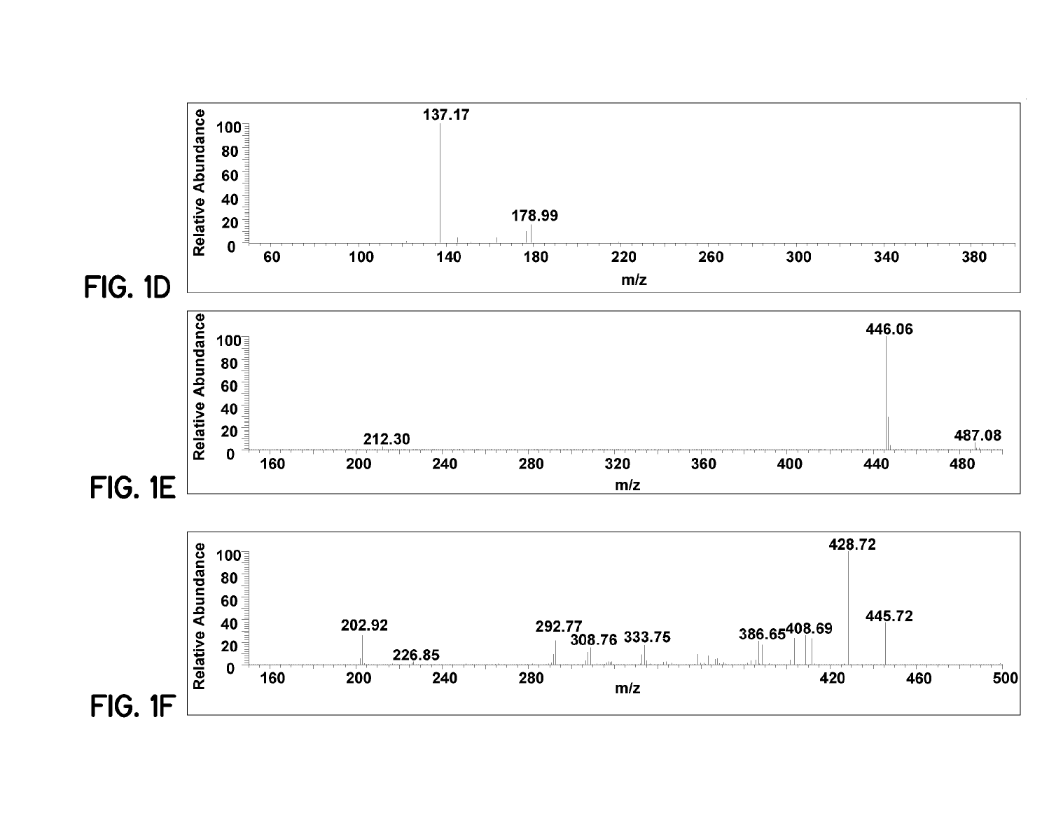

FIGS. 1A to 1F, show the mass spectrometric analysis of curcumin, THC, and OSU-Arg. The respective straight solutions of curcumin, THC and OSU-Arg (10 μg/mL) in 50% acetonintrile containing 0.1% formic acid were infused directly into the mass spectrometer in the positive ion electro-spray ionization mode. The observed full scan mass spectrum showed prominent protonated molecular ions MH⁺ at m/z 369.1 for curcumin (FIG. 1A), m/z 373.0 for tetrahydrocurcumin (FIG. 1C), and m/z 446.0 for OSU-Arg (FIG. 1E), respectively. The MH⁺ ions of curcumin and OSU-Arg were subjected for collision induced dissociation using Argon as the collision gas at an average collision energy of 30%. These ions underwent significant fragmentation to form prominent products ion at m/z 177.0 (FIG. 1B), 137.0 (FIG. 1D), and 429.0 (FIG. 1F), for curcumin, THC, and OSU-Arg, respectively. The mass and tandem mass spectra of curcumin and THC is consistent with literature values. Therefore, the following three transitional ion channels: m/z 369.1>m/z 177.0, m/z 373.0>m/z 137.0, and m/z 446.0>m/z 429.0, were selected for monitoring curcumin, THC and OSU-Arg, respectively.

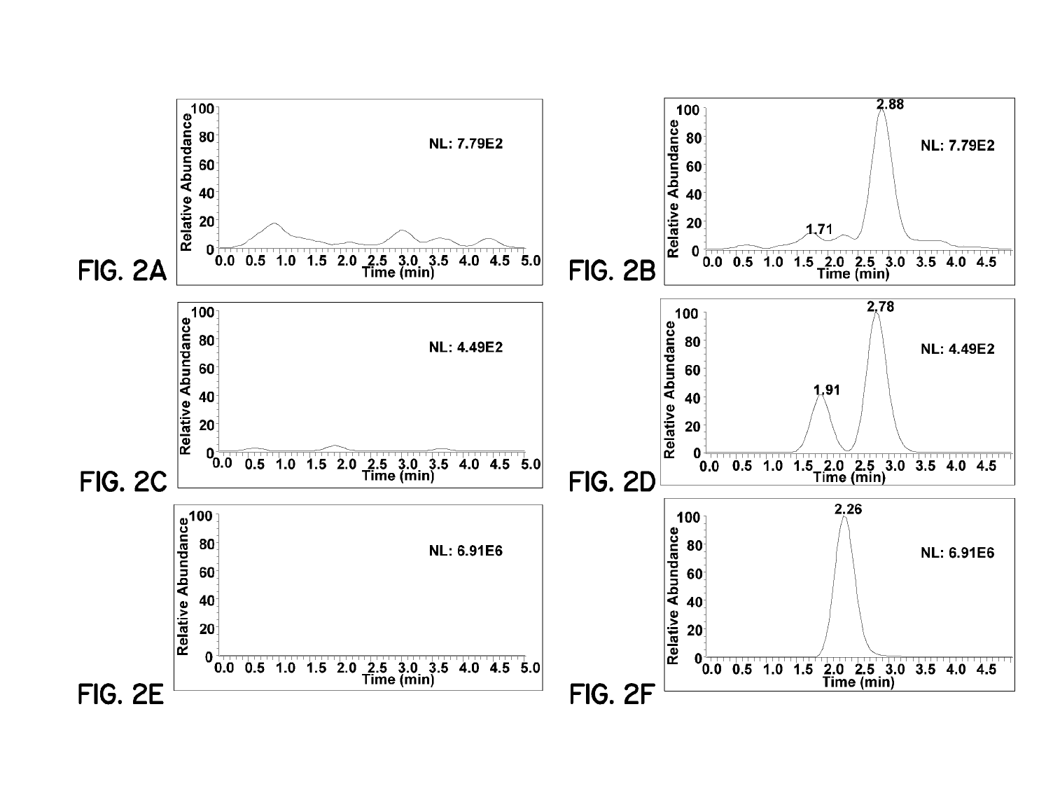

FIG. 2 shows exemplary extracted ion chromatograms of curcumin [m/z 369.0>m/z 177.0 (E=35%)], THC [m/z 373.0>m/z 137.0 (E=35%)], and OSU-Arg [m/z 446>m/z 429 (E=30%)] in the reconstituted solution of ethyl acetate extraction residue of blank mouse plasma (A, C, E) and mouse plasma spiked with 1 ng/mL curcumin (B), 1 ng/mL THC (D) and 100 ng/mL OSU-Arg (F) in 50% ACN/0.1% formic acid (mobile phase). Due to its hydrophobicity, a C₈ analytical column was used to reduce the potential hydrophobic interaction so that the analytes can be eluted in a short time. The extract ion mass chromatograms (XIC) of curcumin, THC and internal standard OSU-Arg obtained in the positive ion LC-ESI-MS/MS analysis are presented in FIG. 2. There is one peak with the retention time of 2.28 min in the XIC of OSU-Arg; however, there are two peaks with retention time of 1.81 min and 2.78 min in the XIC of THC and one peak at 2.88 min for curcumin at its lower limit of quantification (LLOQ, 1 ng/mL), however, there are also two peaks for curcumin at its relative higher concentrations (Data not shown). These results demonstrated that curcumin and THC can be baseline separated from the internal standard (I.S.) under the HPLC condition. The presence of two peaks in the XIC of curcumin and THC is consistent with the existence of two tautomers of curcumin and THC (diketone and keto-enol form), respectively. To test this hypothesis, a curcumin analog: tetramethylcurcumin (FIG. 1), which can only exist as the diketone form, was prepared according to the literature method (Takeuchi, T., et al., 2006, Genes Cells 11: 223-235). Only a single peak has been observed in its chromatogram (Data not shown). Hence, for the calibration and its stability and pharmacokinetic studies, the sum of the two peak areas was used.

We next developed and validated the method for recovering and measuring curcumin from plasma. Due to its high hydrophobicity, curcumin can easily be extracted using organic solvents, such as ethyl acetate, chloroform, and dichloromethane. To avoid the potential carcinogenicity of chloroform and methylene chloride, ethyl acetate was used for curcumin extraction from mouse plasma. Curcumin showed a linearity in the region of 1 to 1000 ng/mL with a linear regression coefficient greater than 0.99. The extraction recoveries were calculated to be about 48-60% at the concentration of 5, 50 and 500 ng/mL in mouse plasma. The matrix effects were quantitatively compared to the solutions prepared in the mobile phase (85%-101%). The results showed that the mass signal of curcumin was not significantly affected by the matrices. The method was then validated at 1, 5, 50 and 500 ng/mL concentrations with six replicates of samples and the data showed acceptable within day relative standard deviations. As shown in Table 2, the within-day and between-day validation parameters: the CVs of all evaluated quality control concentrations for both within-day and between-day were below 15% and the within-day accuracy was in the range of 85% to 105%, which are within the criterion of an validated FDA GLP analytic method. Therefore, the method is suitable for quantitative determination of curcumin and THC from mouse plasma.

TABLE 2
The between-day and within-day validation parameters of
curcumin and THC in mouse plasma.

| Conc. | Curcumin | | | THC | | |
|---|---|---|---|---|---|---|
| ng/ | Accu- | C.V. | C.V. | Accu- | C.V. | C.V. |
| mL | racy | (Within) | Between | racy | (Within) | Between |
| 1 | 104.37 | 11.68 | 11.71 | 86.8 | 7.33 | 13.9 |
| 5 | 88.6 | 7.35 | 7.01 | 90.3 | 5.64 | 5.36 |
| 50 | 85.0 | 14.70 | 3.50 | 100.2 | 3.25 | 1.94 |
| 500 | 100.2 | 9.96 | 4.96 | 109.9 | 5.92 | 7.51 |

We also evaluated the stability of curcumin in mouse plasma and in an autosampler. The in-vitro stability of curcumin of 5, 50, 500 ng/mL was evaluated in the mouse plasma at 37° C. and the reconstituted solution in auto-sampler at 4° C. The stability of curcumin in mouse plasma indicated that more than 97% curcumin is decomposed in 2 hours following incubation and the compound was below detection limit (1 ng/mL) by 4 hour. The half life of curcumin in mouse plasma was 23.6 min. Therefore, all mouse blood and plasma samples have to be processed on ice to avoid potential degradation for its pharmacokinetic studies. Curcumin in the reconstituted solution in the auto-sampler at 4° C. is quite stable for up to 8 hours (>85% of its original concentrations).

To extract curcumin and the internal standard (I.S.) OSU-Arg from mouse plasma, ethyl acetate was used as follows: a 100 μL aliquot of blank mouse plasma spiked with various concentrations of curcumin, THC, and a fixed concentration of OSU-Arg (100 ng/mL) were extracted with 1.0 mL ethyl acetate. The ethyl acetate layer was collected and dried under a mild stream of nitrogen. The residues were then reconstituted with 100 μL 50% acetonitrile and 0.1% formic acid. The resulting solution was then analyzed by the LC-MS/MS method.

To evaluate the matrix effect and recovery of curcumin in mouse plasma using ethyl acetate extraction, three separate batches of curcumin and THC samples at concentrations of 5, 50 and 500 ng/mL were prepared. The first batch was prepared by spiking curcumin and THC in mobile phase B (MP-B). The second batch was prepared by spiking curcumin and THC in the reconstitution solution of ethyl acetate extract residues of blank mouse plasma in MP-B, and the third batch was prepared as the reconstitution solution of ethyl acetate extract residues of curcumin and THC-spiked mouse plasma in MP-B. The recovery of curcumin and THC was calculated by the peak area ratios of curcumin and THC to the I.S. of the second batch samples to those of the third batch samples. The matrix effect of curcumin and THC was evaluated by the peak area ratios of the second batch to those of the first batch samples.

To validate the assay, a 10 μL aliquot of the solutions of 10-10,000 ng/mL curcumin or THC was spiked into 100 μL mouse plasma containing a constant amount of OSU-Arg to make a final concentration of 1.0 to 1000 ng/mL. The samples were extracted using ethyl acetate according to the protocol described above. The within-run precision values were determined in six replicates at concentrations of 1.0, 5.0, 50 and 500 ng/mL and the between-run precision was determined across these concentrations in six different days. The mean concentration and the coefficient of variation (CV) were calculated as the relative standard deviation (%) from the six replicates. The within-day accuracy of the assay was determined by comparing the corresponding calculated mean concentration with the nominal concentration. These samples were injected into LC for LC-MS/MS analysis.

Next several formulations of curcumin were prepared according to the following procedure, Curcumin (1.65 g) powder was added into 10 mL PEG 400 or PEG 600 (Sigma, St. Louis, Mo. USA) in a glass tube covered by aluminum foil. After stirring, the tube was heated at 100° C. in a heating block for 15 min until the curcumin powder was completely dissolved in PEG 400 or PEG 600. After cooling the tubes at room temperature for about 10 minutes, various amounts of Cremophor® EL, Tween® 80, medical ethanol (USP), and/or sesame oil were added into the tube and the mixture was mixed by vortex. The final concentration of curcumin of these seven formulations is 100 mg/mL.
Formulation F1. 100 mg/mL curcumin PEG 400 solution;
Formulation F2 to F5: 100 mg/mL curcumin PEG 400 and Cremophor® EL solution (v/v: 4:1; 2:1; 4:3; and 1:1, respectively);
Formulation F6: 100 mg/mL curcumin PEG 400, Cremophor® EL and Tween® 80 solution (v/v/v 4:2:1); and
Formulation F7: 100 mg/mL curcumin PEG 600 and Cremophor® EL solution (v/v 2:1).

The pharmacokinetics of gel curcumin formulations and suspension curcumin formulation was evaluated. CD2F1 mice (˜20 g) (Harlan, Indianapolis, Ind.) were used in this study. All animal procedures were performed according to a protocol in compliance with The Ohio State University Laboratory Animal Resources (ULAR) policies, which adhered to the guideline and “Principles of Laboratory Animal Care by National Institutes of Health. For formulation optimization, gavage administration of approximately 250 μL (adjusted by body weight and doses) curcumin formulations results in a dose of 1000 mg/kg in mice. For complete pharmacokinetics analysis, administration of approximately 450 μL (adjusted by body weight and doses) formulation F7 by gavage resulted in a dose of 1800 mg/kg. The blood was collected by cardiac puncture under CO₂ anesthesia 1 hour post-administration for formulation optimization and the following time schedule of 0 (pre-dose), 0.25, 0.5, 1, 2, 3, 4, 6, 8 and 24 hours after dosing was collected. The blood samples in the heparinized tubes were centrifuged at 1000 g for 5 minutes in a 4° C. micro-centrifuge and the supernatant of each was collected and kept at −80° C. until analysis. The curcumin levels in plasma were measured using the LC-MS/MS assay. Plasma concentration-time data were analyzed by the WinNonlin computer software (Pharsight 5.0, Mountain View, Calif.) using appropriate pharmacokinetic models.

The curcumin in mouse plasma was analyzed. An aliquot of 100 μL of pharmacokinetic plasma samples from gel curcumin studies (10× dilutions with blank plasma) and from suspension curcumin formulations studies was mixed with 10 μL internal standard (10 μg/mL stock in 50% acetonitrile). The resulting solution was extracted and processed according to the extraction protocol described above. An aliquot of 10 μL of the final solution was injected into LC for LC-MS/MS analysis.

The curcumin formulations were optimized to improve the bioavailability of curcumin in plasma. Earlier developed emulsions of curcumin, such as a recently reported self-microemulsifying drug delivery system (SMEDDS) curcumin have a relatively low loading capacity of curcumin (20 mg/mL) limiting the characterization of the pharmacokinetics of those compositions. [See Cui, J. et al., Int J. Pharm. 2009 Apr. 17; 371(1-2):148-55]. Thus, the first challenge to overcome was to increase the curcumin loading capacity of the present formulations to allow for pharmacokinetic evaluation of the compositions. To achieve this goal, the solubility of curcumin was evaluated in PEG 400 at different temperatures (shown in Table 1). It was found that about 200 mg curcumin can dissolve in 1 mL PEG 400 at 100° C. and mass spectrometric analysis of these solution demonstrated that curcumin is quite stable (Data not shown). Then, a phase diagram of PEG 400 or PEG 600, two emulsifier or surfactants: Tween® 80, Cremophor® EL or the mixture, and a vegetable oil sesame oil was reconstituted (data not shown) to determine their miscible region.

Based on the miscible region, several formulations (F1 to F7) of curcumin in the aforementioned solvents were prepared, as described above. Formulation F1: 100 mg/mL curcumin PEG 400 solution; Formulations F2-F5: 100 mg/mL curcumin PEG 400 and Cremophor® EL solution (v/v: 4:1; 2:1; 4:3; and 1:1, respectively); Formulation F6: 100 mg/mL curcumin PEG 400, Cremophor® EL and Tween® 80 solution (v/v/v 4:2:1); and Formulation F7: 100 mg/mL curcumin PEG 600 and Cremophor® EL solution (v/v 2:1).

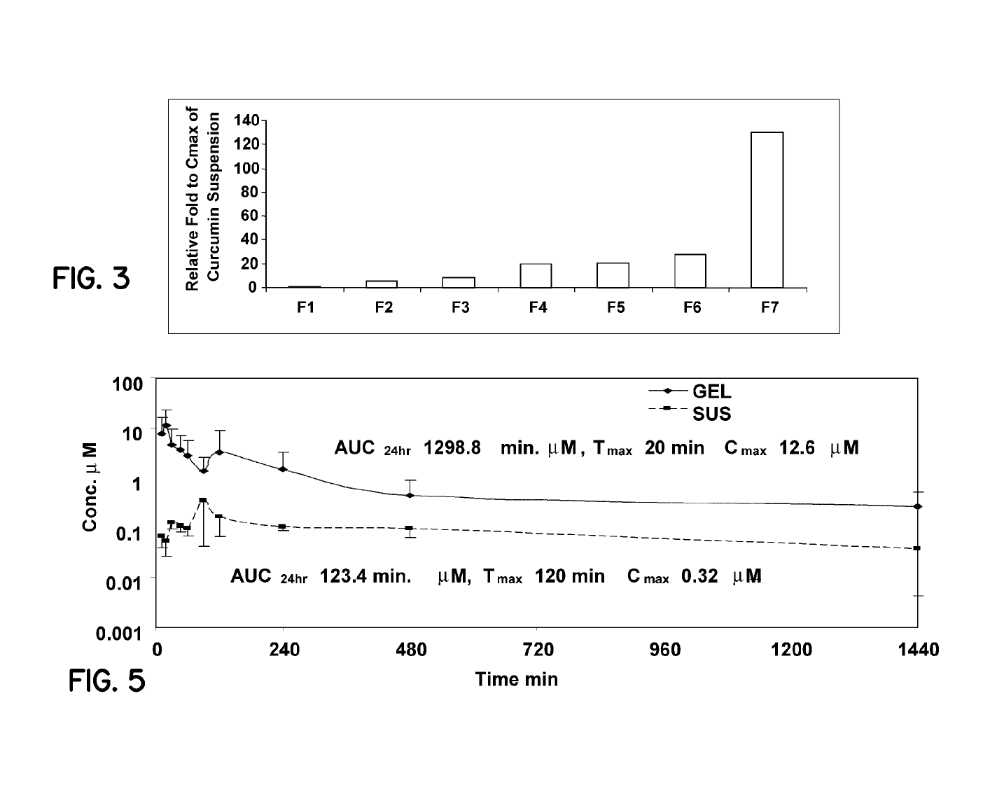

Then, these formulations F1 to F7 as 100 mg/mL curcumin solution were orally administrated at a volume of 0.25 mL resulting in a dose of 1000 mg/kg curcumin to mice with an average 25 g body weight. The plasma level of curcumin was determined 1 hour after its dose, since previous pharmacokinetic studies of oral dosing of curcumin in mice showed a Tₘₐₓ in the range of 1 to 2 hour. Additionally, our pilot study of an oral administration of 800 mg/kg curcumin in dimethylsulfoxide (DMSO) showed that the plasma level of curcumin was about 3 μM at the 1 and 2 hour timepoints. As shown in FIG. 3, the plasma level of curcumin in mice after an oral dose of formulation F1 was the lowest and the highest was that of formulation F7. The plasma levels of curcumin of formulation F2 to F5 were higher than that of formulation F1, which suggests that Cremophor® EL can facilitate an increase curcumin's absorption. No significant difference of the plasma level of curcumin between formulation F5 and formulation F6 was found, suggesting that Tween® 80 showed similar function to that of Cremophor® EL. The significant difference between formulations F6 and F7 suggests that PEG 600 can significantly enhance curcumin's absorption as compared to that of PEG 400. As shown in FIG. 3, the plasma level of curcumin after oral administration of formulation F7 is about 6 fold higher than that of formulation F6 in mice.

The aforementioned results demonstrate that both PEG 400 and PEG 600 may enhance the absorption of curcumin in mice; however, PEG 600 can enhance even more (about 5.4 fold) as indicated by higher plasma levels, and adding Cremophor® EL may further enhance its absorption about 4-5 fold. Therefore, four formulations of curcumin in PEG 600 and Cremorphor EL were prepared, as described below. Formulations containing 125 mg/mL curcumin in PEG 600 and Cremophor® EL solution (v/v: 4:1; 2:1; 4:3; and 1:1, respectively) were prepared. Then, these formulations as 200 mg/mL curcumin solution were orally administrated at a volume of 0.5 mL resulting in a dose of 2500 mg/kg curcumin to mice with an average 25 g body weight. The plasma level of curcumin was determined 1 hour and 2 hours after administering the dose, since previous pharmacokinetic studies of oral dosing of curcumin in mice showed a Tₘₐₓ in the range of 1 to 2 hour. As shown in Table. 3, the plasma level of curcumin in mice after an oral dose of these formulations is very high and the inclusion of cremophor EL increases the plasma level of curcumin and also shortens the time to reach Cmax from 2 hr to 1 hr.

TABLE 3
The plasma level of curcumin after oral dose of 2.5 g/kg curcumin
formulated in the following solvents/emulsifiers.

| Formulation | Conc. (μM) |
|---|---|
| PEG 600-1 hr | 10.098 |
| PEG 600-2 hr | 0.368 |
| 80% PEG 600 + 20% Cremophor ® EL-1 hr | 11.484 |
| 80% PEG 600 + 20% Cremophor ® EL-2 hr | 45.011 |
| 66.7% PEG 600 + 33.3% Cremophor ® EL-1 hr | 43.013 |
| 66.7% PEG 600 + 33.3% Cremophor ® EL-2 hr | 16.406 |
| 57% % PEG 600 + 43% Cremophor ® EL-1 hr | 22.880 |
| 57% PEG 600 + 43% Cremophor ® EL-2 hr | 11.573 |
| 50% PEG600 + 50% Cremophor ® EL-1 hr | 54.721 |
| 50% PEG600 + 50% Cremophor ® EL-2 hr | 5.264 |

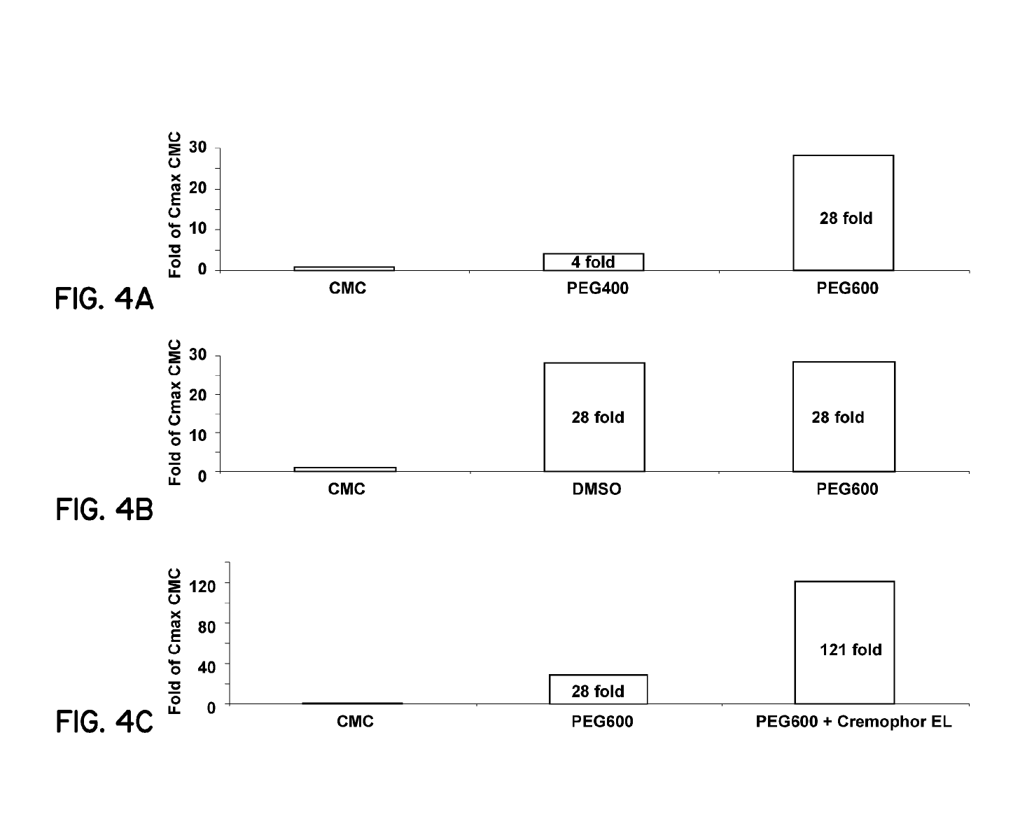

Based on the data above, and the results from a pilot study using curcumin dissolved in DMSO, the enhancement of plasma levels facilitated by these solvent, co-solvent/surfactant/emulsifiers is presented in FIG. 4.

With the above preliminary data, a formulation of curcumin in 66.7% PEG 600+33.3% Cremophor® EL was selected and a full pharmacokinetic analysis of this pharmaceutical gel formulation in comparison to curcumin suspension in carboxylated methylcellulose (CMC) was conducted. In addition, curcumin and its natural analogs and metabolites present in curcumin were also estimated and the results are shown in the following tables and the discussion is provided in the sections below.

After optimization of gel curcumin as 100 mg/mL curcumin formulated in a volume ratio 2:1, PEG 600 and Cremophor® EL, a full-set pharmacokinetics of this formulation and curcumin suspension was characterized at the oral dose of 1800 mg/kg in CD2F1 mice using the validated LC-MS/MS method. The mean plasma concentration-time profiles of curcumin are shown in FIG. 5, and the resulting relevant pharmacokinetic parameters obtained from each of the formulation are listed in Table 4. As determined by this method, the AUC_{0→24hr} of curcumin gel was 1299 μM·min, while the AUC_{0→24hr} of curcumin suspension was 123.4 μM·min. This resulted in a 10.5 fold increased relative bioavailability (FIG. 5) for the gel formulation. These results also demonstrated that Cmax values of 12.6 μM for curcumin gel, 39.4 times higher as compared to 0.32 μM for the suspension curcumin. The evidence suggests that the gel curcumin can deliver in-vitro effective plasma level of curcumin after its oral administration in mice.

TABLE 4
Relevant pharmacokinetics parameters for gel curcumin
(Gel) and suspension curcumin (SUS) in Mice.

| Parameter | Units | SUS | Gel |
|---|---|---|---|
| λ_z^{a} | 1/min | 0.0009 | 0.0017 |
| HL_λ_z^{b} | min | 776.05 | 413.25 |
| Tmax^{c} | min | 90.00 | 20.0 |
| Cmax^{d} | μmol/L | 0.353 | 12.60 |
| Clast^{e} | μmol/L | 0.037 | 0.480 |
| AUClast^{f} | min*μmol/L | 123.64 | 1298.8 |
| Vz_F_obs^{g} | L | 33.91 | 1243.13 |
| Cl_F_obs^{h} | L/min | 0.0303 | 2.085 |
| AUMClastⁱ | min*min*μmol/L | 60397.9 | 718777.9 |
| MRTlast^{j} | min | 488.5 | 340.4 |

^{a}elimination rate;
^{b}the half life of terminal elimination;
^{c}time to reach the maximal plasma concentration;
^{d}The maximal plasma level;
^{e}the plasma level of the last time point,
^{f}the area under the concentration-time curve to the last time point;
^{g}volume distribution;
^{h}the clearance,
ⁱthe area under the moment curve to the last time points;
^{j}mean residence time

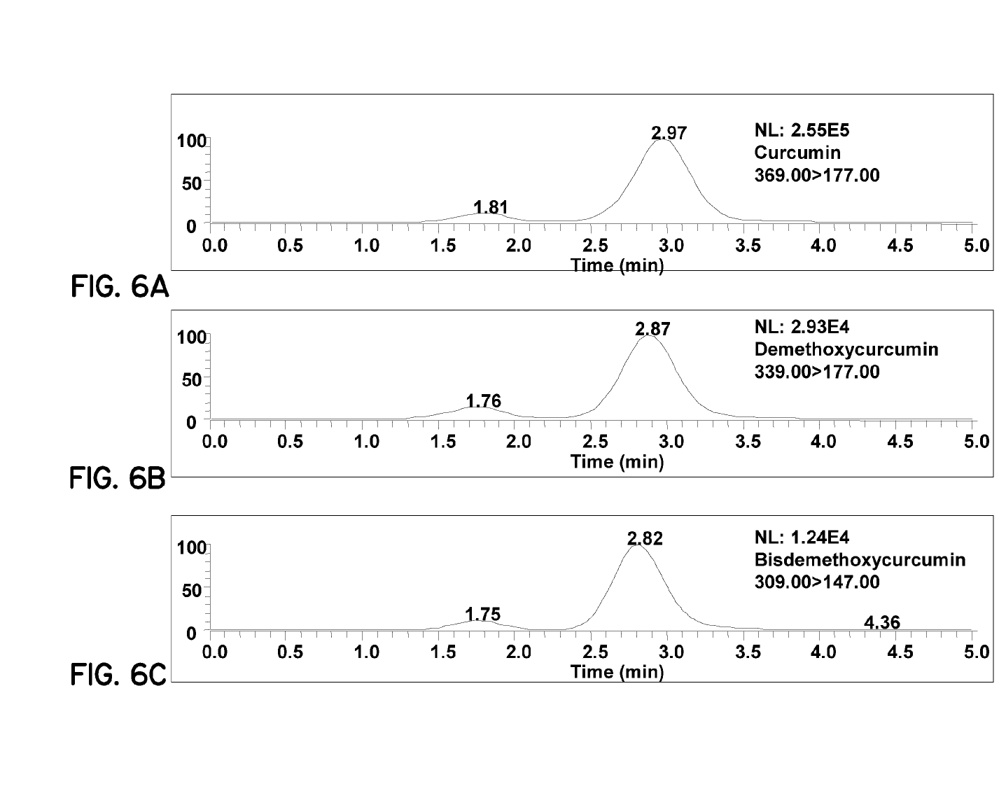

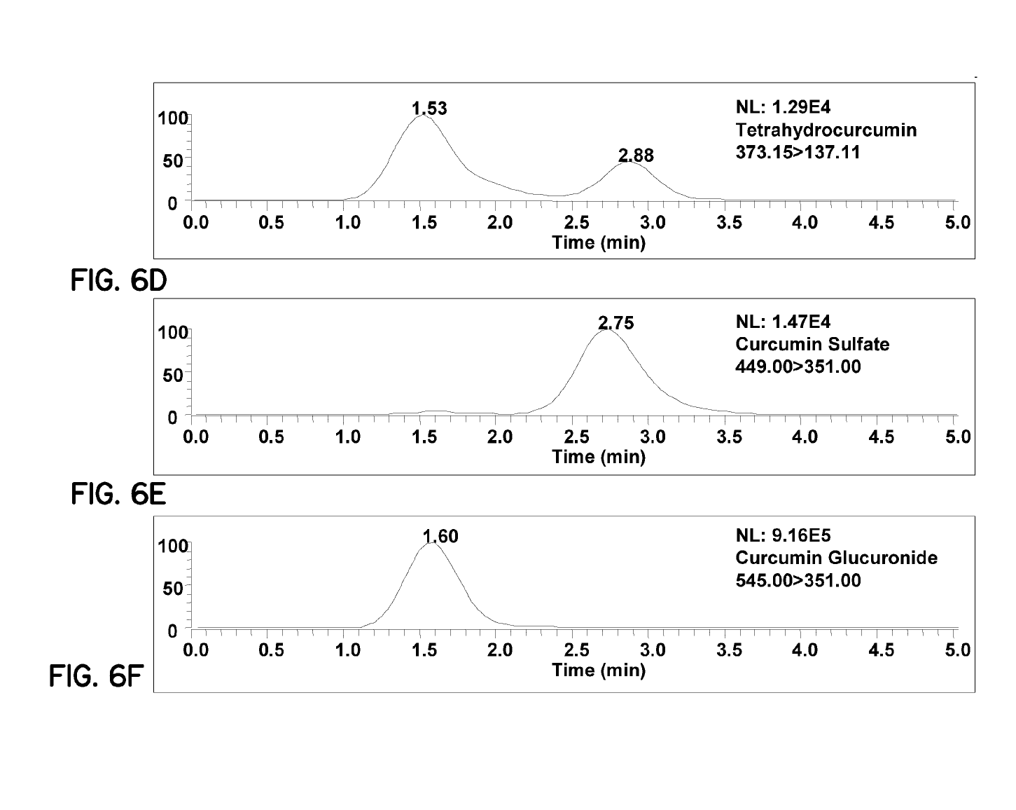

Identification of curcumin metabolites in plasma of samples following fifteen minutes after oral administration of gel curcumin to mice was performed. Extracts of bio-matrices were subjected to HPLC mass spectrometric analysis. Based upon specific MRM transitions (in parenthesis), the analysis showed unambiguously the presence of curcumin (369>177, FIG. 6A), desmethoxycurcumin and bisdesmethoxycurcumin (339>177, FIG. 6B; and 309>147, FIG. 6C, respectively), two curcuminoids co-extracted with curcumin from the curcuma plant, and curcumin metabolites, curcumin sulfate (449>351); curcumin glucuronide (545>351); and tetrahydrocurcumin (371>137), in mouse plasma (FIGS. 6D, 6E, 6F, respectively). The occurrence of these species has previously been suggested in blood mouse, which received suspension curcumin Therefore, the results suggest that gel curcumin does not confound the qualitative pattern of curcumin metabolism in vivo.

Several factors have been proposed to contribute the low bioavailability of curcumin including poor absorption and rapid metabolic elimination. Previous study demonstrated that suspending curcumin in a gel can enhance its absorption. Recently, it was found that Cremophor® EL can inhibit the glucuronidation of raloxifene and metabolism study of curcumin in the mouse demonstrated that most of curcumin was converted to its glucuronide as a major metabolite (more that 99 fold of the curcumin concentration in plasma), and to its sulfate and tetrahydrocurcumin or other reduced curcumin as minor metabolites after its oral administration. Therefore, without be held to any particular theory, the Cremophor® EL may inhibit the glucuronidation of curcumin resulting in enhanced circulation plasma levels.

TABLE 5
The plasma levels of curcumin (Cur) and curcumin natural analogs and
metabolites in mouse after oral dose of 1.8 g/kg of curcumin formulation
in 66.7% PEG 600 + 33.3% Cremophor ® EL.

| Time (min) | CUR (ng/mL) | DMO^{a} | BDMO^{b} | THC^{c} | GLU^{d} | SUL^{e} |
|---|---|---|---|---|---|---|
| 1440 | 20.05343 | 3.838479 | 0 | 2.238948 | 24.2129 | 0 |
| 480 | 58.29402 | 15.36469 | 10.92166 | 4.671712 | 716.5084 | 4.049819 |
| 240 | 169.2053 | 40.41871 | 34.52579 | 8.173343 | 1243.665 | 9.948785 |
| 120 | 231.4067 | 39.46973 | 15.39398 | 8.630952 | 1070.25 | 7.752384 |
| 90 | 134.6173 | 29.13655 | 11.08493 | 6.037541 | 1336.072 | 8.636514 |
| 60 | 448.4571 | 76.53878 | 37.32574 | 39.22903 | 2057.704 | 18.74807 |
| 45 | 660.6012 | 118.2951 | 66.91855 | 62.50282 | 2539.161 | 27.51175 |
| 30 | 2451.63 | 108.128 | 51.41495 | 219.0996 | 5469.386 | 43.06047 |
| 20 | 1620.253 | 224.153 | 115.1845 | 238.2446 | 4428.71 | 54.83276 |
| 10 | 1621.207 | 147.9478 | 65.72002 | 112.6977 | 3083.366 | 29.44048 |

^{a}DMO: desmethoxycurcumin,
^{b}BDMO, bisdesmethoxycurcumin,
^{c}THC, tetrahydrocurcumin,
^{d}GLU: curcumin O-monoglucuronide,
^{e}SUL: curcumin O-sulfate

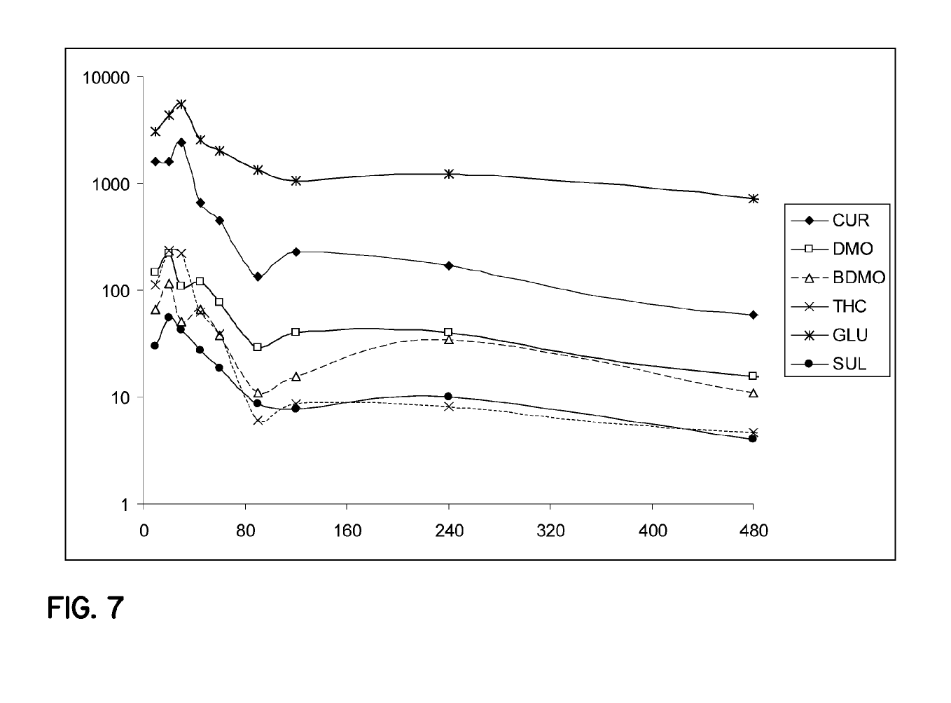

FIG. 7 shows the plasma concentration-time profiles of Curcumin (CUR), demethoxycurcumin (DMO), bisdemethoxycurcumin (BDMO), tetrahydrocurcumin (THC), curcumin O-glucoronide (GLU), curcumin O-sulfate (SUL) in mouse after oral administration of 1.8 g/kg curcumin gel in 66.7% PEG 600±33.3% Cremophor® EL. These data demonstrate that curcumin glucuronide is the predominant circulation form of curcumin followed with curcumin, demethoxycurcumin and bisdemethoxycurcumin and curcumin sulfate. The area under the curve for each species is listed in the Table 6 below.

TABLE 6
The AUC of Cur, DMO, BDMO, THC, GLU, SUL and their relative
percentage in mouse after oral administration of 1.8 g/kg curcumin
gel in 66.7% PEG 600 + 33.3% Cremophor ® EL.

| Species | CUR | DMO | BDMO | THC | GLU | SUL |
|---|---|---|---|---|---|---|
| AUC μM.min | 487.8 | 90.9 | 60.2 | 38.29 | 1863.2 | 16.2 |
| % curcumin | 100.0 | 18.6 | 12.3 | 7.9 | 382.0 | 3.3 |

The content of curcumin, demethoxycurcumin and bis demethoxycurcumin is 85.7%, 11.8% and 4.81%.

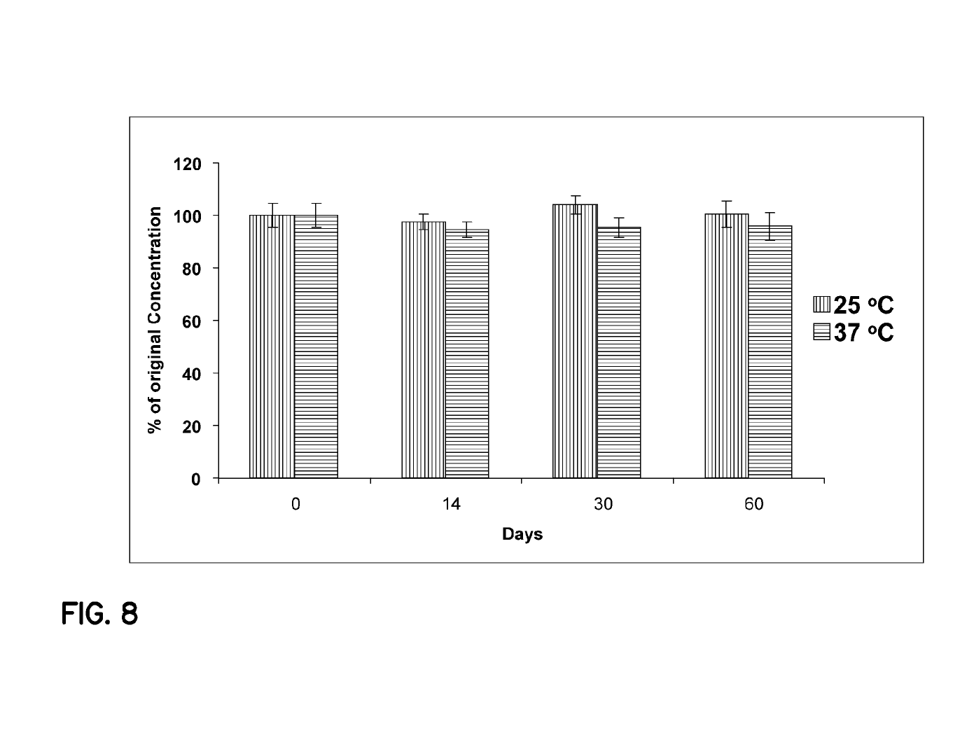

After showing the favorable pharmacokinetics, the stability of this formulation was evaluated as follows: Formulations of curcumin (100 mg/mL) in 66.7% PEG 600 and 33.3% Cremophor® EL were stored at room temperature and at 37° C. (in triplicate). Samples were taken at 0, 14, 30 and 60 days following storage to quantify the concentration of curcumin using UV-VIS spectrometer. The results are shown in Table 7 and FIG. 8. This stability study data suggests that a curcumin formulation in 66.7% PEG 600 and 33.3% Cremophor® EL is stable at room temperature and 37° C. for at least two months.

TABLE 7
The Stability of Curcumin in PEG 600 and
Cremophor ® EL Formulation.

| Time (Days) | 25° C. (% to day 0) | 37° C. (% to day 0) |
|---|---|---|
| 0 | 100 | 100 |
| 15 | 97.4 | 94.4 |
| 30 | 104.1 | 95.3 |
| 60 | 100.6 | 95.8 |

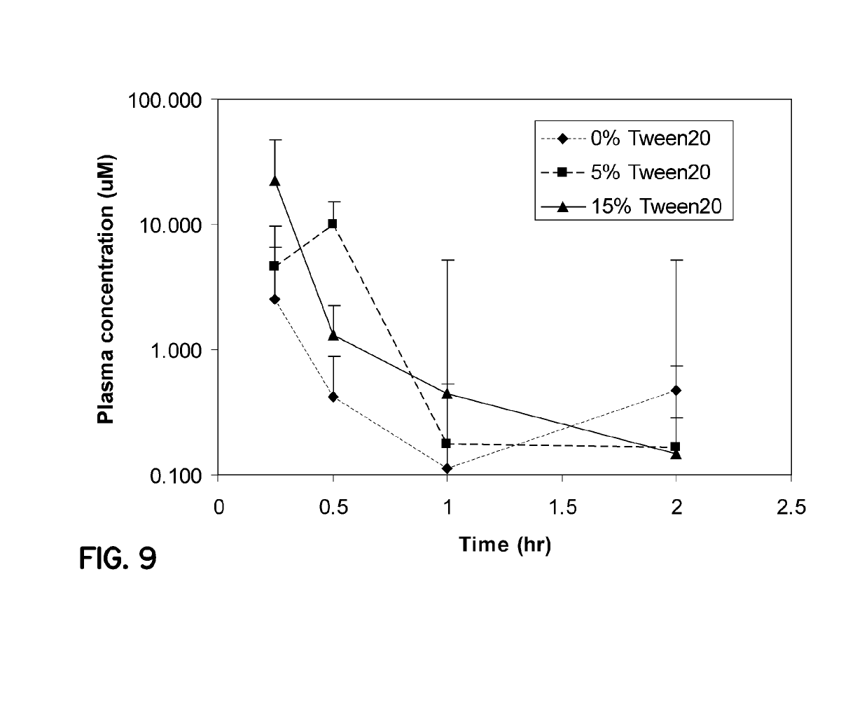

Tween® 20 may enhance the plasma level of curcumin after its oral administration at 1.0 g/kg of curcumin. A pilot pharmacokinetic study of curcumin in various ratios of PEG 600 and Tween® 20 was performed. The plasma concentration of curcumin was tabulated in Table 8 and the plasma concentration-time profile of curcumin at the following time points: 15, 30, 1 and 2 hr are shown in FIG. 9. This result demonstrated that 15% Tween® 20 may increase plasma level of curcumin (22.5 μM), which is 9 fold higher than that of PEG 600 (2.5 μM) alone, which is better than that in Cremophor® EL (5 fold).

TABLE 8
The plasma levels of curcumin (Cur) in mouse after oral dose of 1.0 g/kg
of curcumin formulation in PEG 600 and various Tween ® 20

| | | 0% Tween ® 20 | 5% Tween ® 20 | 15% Tween ® 20 |
|---|---|---|---|---|
| Time (hr) | | (μM) | (μM) | (μM) |
| 0.25 | (Ave ± SD) | 2.526 ± 4.033 | 4.595 ± 7.101 | 22.577 ± 24.940 |
| 0.5 | (Ave ± SD) | 0.422 ± 0.454 | 10.048 ± 15.220 | 1.318 ± 0.954 |
| 1 | (Ave ± SD) | 0.112 ± 0.063 | 0.177 ± 0.191 | 0.447 ± 0.092 |
| 2 | (Ave ± SD) | 0.476 ± 0.274 | 0.166 ± 0.130 | 0.148 ± 0.135 |

Next, we compared the ratio of the AUC of curcumin and its metabolites in mice after oral administration of gel curcumin. It was found that the ratio of the AUC of curcumin glucuronide is only about 4 fold of that of curcumin, which is significantly lower than that previous reported (more than 99% of circulating curcumin as its glucuronides).

Therefore, the curcumin gel formulation was optimized based on the plasma level of curcumin one hour after oral administration to mice. An improved gel curcumin formulation with a curcumin concentration of 100 mg/mL was established, which had a 5 fold increase over the loading capacity reported by Cui, J. et al., Int J. Pharm. 2009 Apr. 17; 371(1-2):148-55. Additionally, the co-solvent PEG 400 was replaced by PEG 600, since comparison study of formulation F6 and F7 demonstrated that 1 hr post-dose plasma level of curcumin with formulation F7 (PEG 600) was about 6 fold than that with curcumin formulation F6 (PEG 400).

To our knowledge, this is the first demonstration of matching an in-vitro effective concentration in-vivo (20 μM) following oral administration of curcumin based on the previous published studies. Thus, a full-set of pharmacokinetics of this curcumin gel was characterized and compared with its suspension with a capacity of dosing the curcumin gel as a single dose at 1800 mg/kg. A more than 10 fold increased in oral bioavailability and about 40 fold increase of curcumin level in plasma were documented for this gel relative to the suspension. Metabolic profile and semi-quantification of three putative hypomethylating curcumin metabolites suggest that the gel did not alter the qualitative metabolic profile of curcumin, but lower the ratio of curcumin glucuronidation to curcumin in plasma. This may implicate potential inhibition of curcumin glucuronidation by Cremophor® EL, which may contribute to its enhanced bioavailability.

### Example 2

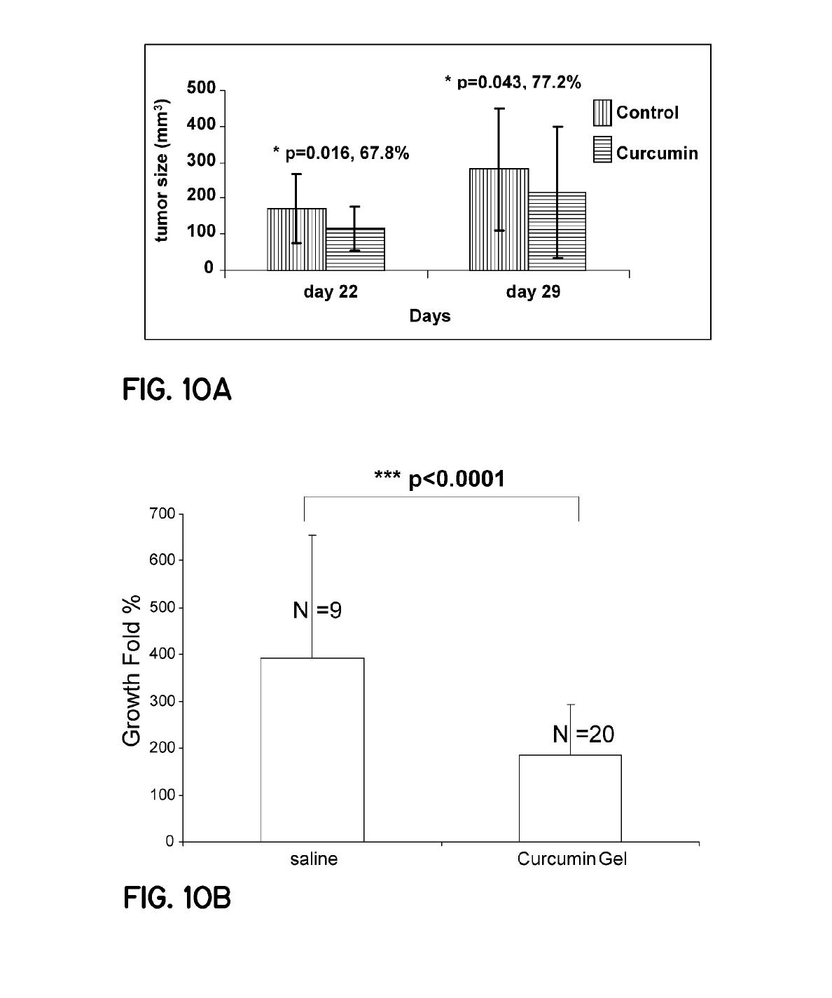

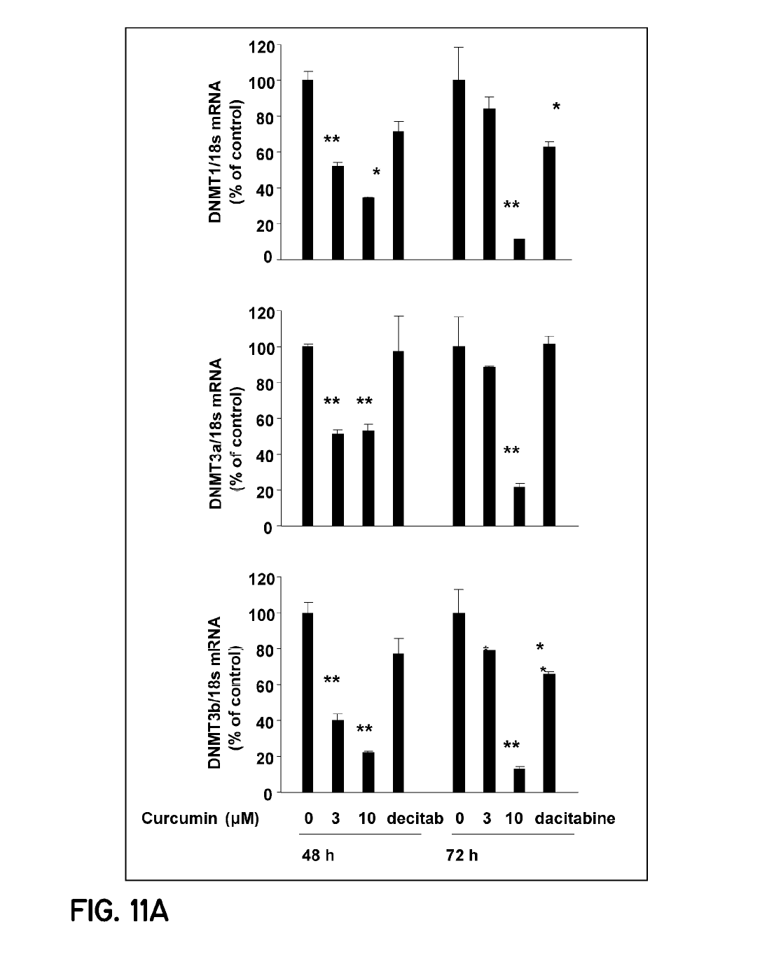

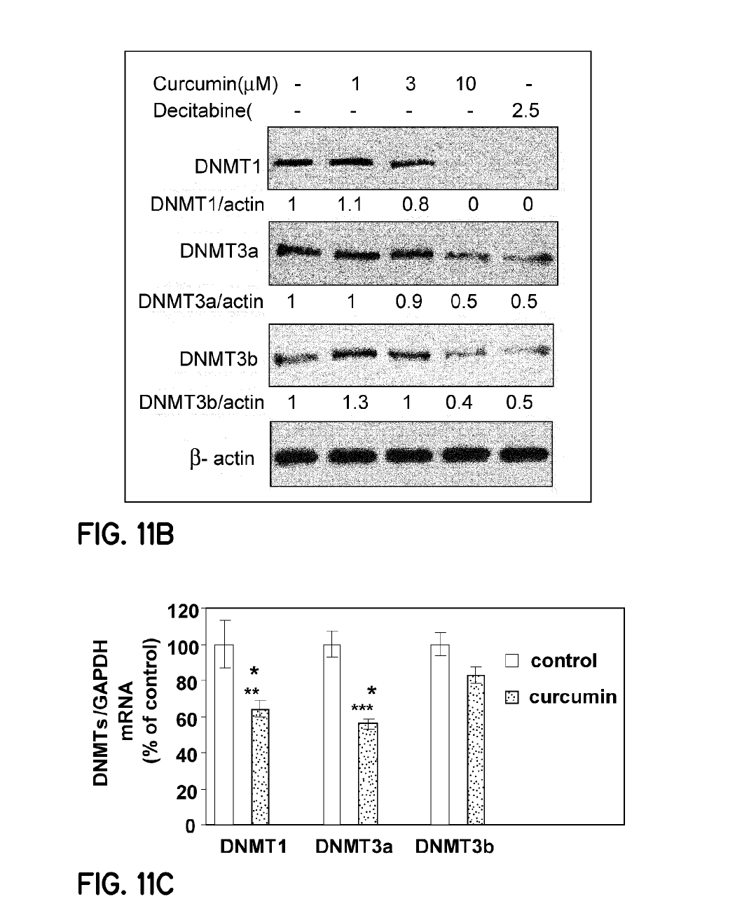

Curcumin formulations according to embodiments of the invention has anti-tumor activity. For these studies, the curcumin gel formulation was tested in a breast cancer model using MDA-MB-231 breast cancer cell engrafted nude mice. Female athymic nu/nu mice (4-6 weeks old, 18-22 g) were obtained from Charles River Laboratory (Wilmington, Mass.) and acclimated for 1 week in a pathogen-free enclosure before start of study. Animals were given sterile rodent chow and water ad libitum and were housed in sterile filter-top cages with 12 hour light/dark cycles. All experiments were conducted in accordance with the guidelines of the Association for Assessment and Accreditation of Laboratory Animal Care International (AAALAC). MDA-MB-231 cells (5×10⁶ cells per mouse) were suspended with cell culture medium, and subcutaneously implanted into the right flank of the athymic nu/nu mice. When tumors were grown between 100 to 200 mm³, treatments were initiated. Mice were randomly assigned into two cohorts for the anti-tumor growth activity studies, with various number of mice per group. Curcumin formulated as curcumin gel was orally given at the dose of 300 mg/kg everyday for 12 days. Also, curcumin was given intraperitoneally as a solution of a mixture of DMSO: ethanol:saline (10:3:7) at the dose of 100 mg/kg daily for five days a week, and the placebo formulation was used as a control for 4 weeks. Tumor volume was calculated by using the equation V=2*A*B²/3, where A is the longer diameter (mm) and B is the shorter diameter (mm) and expressed in mm³. After 4 weeks, mice were euthanized and tumor tissues were weighed and collected for analysis. The summary of this study is shown in FIG. 10A [Control: DMSO:ethanol:saline; Curcu (i.p. curcumin solution)] and FIG. 10B. [Control as non-treatment and Gel Curcu (Curcumin Gel)].

As shown in FIG. 10A, the tumor size decreased 32.2% relative to the control (p=0.016, two sample t-test, n=9) at 22 day and 22.8% (p=0.047, two sample t-test, n=9) at 29 day treatment with curcumin. This result demonstrated that curcumin can prevent the MDA-MB-231 cell engrafted tumor development and growth.

As shown in FIG. 10B, the tumor growth rate decreased 52.7% relative to the control (p=0.0001, two sample t-test, n=9 for control and n=20 for curcumin gel) at 12 day. This result demonstrated that curcumin gel can also significant inhibitor of MDA-MB-231 cell engrafted tumor growth in nude mice. The actual number demonstrated that oral administration of 300 mg/kg curcumin gel seems to be more potent than that of i.p. administration of 100 mg/kg curcumin (p=0.017, n=9 for i.p. dose and n=20 for oral dose).

### Example 3

Abnormal DNA methylation has been observed in some types of cancers. As such, we evaluated the effect of the curcumin formulations according to embodiments of the invention on DNA methylation.

Materials: Decitabine (DAC) was obtained from the National Cancer Institute and used without further purification. Curcumin, methanol, acetonitrile (HPLC grade), ammonium formate, ammonium acetate, ammonium bicarbonate, 5-methyl-2-deoxycytidine (5mdC), 2-deoxycytidine (2dC), 2-deoxyguanosine (2dG), nucleophosphatase (NP1), snake venom phosphatase (SVP), and alkaline phosphatase (AP), deoxynucleotide triphosphate (2.5 mM), AmpliTaqGold polymerase and 10×PCR buffer were purchased from Sigma-Aldrich (St. Louis, Mo.). The primers for amplification of p15^{INK4B} and its bisulfite-converted promoter region, p21, DNMT1, DNMT3a and DNMT3b, and Sp1 binding promoter region in DNMT1 were purchased from either Sigma-Aldrich (St. Louis, Mo.) or Integrated DNA Technology (IDT, Coralville, Iowa). M. SssI methylase, s-adenosyl-methionine (SAM) (3.2 mM) and 10× incubation buffer were purchased from New England Biolab, Inc. (Beverly, Mass.). The HPLC-grade water (>18 mΩ) was obtained from an E-pure water purification system (Barnstead, Dubuque, Iowa). Antibodies to DNMT1 was purchased from New England Biolabs (Ipswich, Mass.), to DNMT3a, and DNMT3b, Sp1, NF-κB p65, p50 and Histone 2B (H₂B) from Santa Cruz (Santa Cruz, Calif.), and to β-actin from Aldrich-Sigma (St. Louis, Mo.).

Cell Cycle and Apoptosis Analysis: MV4-11, ML-1, HL-60 and K562 leukemia cell lines were cultured in RPMI medium (VWR International, Inc., West Chester, Pa.) supplemented with 10% fetal bovine serum (Invitrogen, Carlsbad, Calif.) and 1% (v/v) penicillin/streptomycin (Invitrogen Life Technologies; Carlsbad, Calif.) antibiotic solution at 37° C. in an incubator under 5% CO₂ atmosphere. For the ex-vivo studies, mononuclear cells obtained from bone marrow (BM) of patient with AML were prepared by Ficoll-Hypaque (Nygaard) gradient centrifugation and cultured with serum-free expansion medium (SFEM) (StemCell technologies, Vancouver, BC) supplemented with GM-CSF (50 ng/ml), IL-3 (20 ng/ml), IL-6 (20 ng/ml) and SCF (100 g/ml). These cells were treated with indicated concentrations of curcumin or decitabine (as a positive control) for indicated times. The cell cycle and apoptosis of MV4-11 cells was analyzed on a BD FACS Calibur (Beckman Coulter, Fullerton, Calif.) according to the standard protocol provided by the manufacturer.

Western Blot Analysis:

Cells or tumor tissues were homogenized and lysed in ice-cold lysis buffer (20 mM, pH 7.0 HEPES, 150 mM NaCl, 0.1% NP40 supplemented with 1 mM β-glycerophosphate, 1 mM Na₃VO₄, 1 mM NaF, 1 mM benzamide and 1 mM phenylmethylsulfonyl fluoride) with protease inhibitors (Protease Inhibitor cocktail Set III, Calbiochem-Novabiochem Corporation, La Jolla, Calif.), and incubated on ice for 20 mM. The lysate was centrifuged at 12,000 g for 10 mM at 4° C. Proteins in the supernatants were resolved on 4-15% SDS-polyacrylamide gradient gels, and then transferred onto nitrocellulose membranes, incubated with appropriate antibodies and subjected to Western blotting. The immunoblotted proteins were detected by ECL reagent (GE Healthcare Bio-Sciences Corp., Piscataway, N.J.).

Quantitative Real-Time RT-PCR Assays:

Quantitative RT-PCR was used to quantify the mRNA levels of DNMT1, DNMT3a, DNMT3b, p15_{INK4B} and p21. Total RNA was extracted with Trizol reagent (Invitrogen, Carlsbad, Calif.) and reverse transcribed by reverse transcriptase (Invitrogen). Quantitative real-time PCR reactions were performed in triplicate using Taqman gene expression assay (Applied Biosystems) with an ABI prism 7700 detector (Applied Biosystems). The target gene values were normalized to the values of internal control GAPDH or 18 S, and expressed as relative percent change (100×2^{(˜ΔΔCt)}) over non-treated samples.

DNA Methylation Analysis of the p15^{INK4B} Promoter Region:

Genomic DNA isolated from MV4-11 cells was treated with bisulfite using the Epi methylation Kit (Qiagen, Minneapolis, Minn.) according to the instruction from the manufacturer and followed by PCR amplification as follows. The sequences of p15^{INK4B} primers were: forward 5′-GGG AGG GTA ATG AAG TTG AGT TTA-3′ (SEQ ID NO. 1), and reverse 5′-ACC CTA AAA CCC CAA CTA CCT AAA T-3′ (SEQ ID NO. 2). PCR amplifications were performed as follows: 95° C. for 5.5 min, 55° C. for 30s for 5 cycles, and 52° C. for 30 s for 38 cycles, 72° C. for 30s for 38 cycles, with a final step at 72° C. for 1 min. PCRs were carried out in a 100 μl volume containing 10 μl of buffer (10×), 2 μL of each primer (10 μM), 2 μL of (10 mM) dNTPs, 2 U of Platinum Taq DNA polymerase (Invitrogen, Carlsbad, Calif.), 70.6 μL ddH₂O and 10 μL of bisulfite treated DNA in a GeneAmp 9700 thermal cycler (Perkin-Elmer, Norwalk, Conn.). PCR products were purified using QIAquick columns and eluted in water. A 500 ng aliquot of the PCR product was incubated with 25 UM. SssI and 320 μM of pH 7.9 SAM solutions containing 50 mM NaCl, 10 mM Tris-HCl, 10 mM MgCl₂, 1 mM dithiothreitol and 5 μL NE buffer and H₂O to make up a final volume of 50 μL for 90 min. Fully methylated PCR products were purified using the QIAquick PCR Purification kit (Qiagen, Minneapolis, Minn.), according to the manufacturer's instructions and 200 ng of the purified DNA was hydrolyzed and the concentrations of 5mdC and 2dC in the hydrolysate were measured by LC-MS/MS using the conditions as described previously.

Xenograft Animal Model Study:

Female athymic nu/nu mice (4-6 weeks old, 18-22 g) were obtained from Charles River Laboratory (Wilmington, Mass.) and acclimated for 1 week in a pathogen-free enclosure before start of study. Animals were given sterile rodent chow and water ad libitum and were housed in sterile filter-top cages with 12 hour light/dark cycles. All experiments were conducted in accordance with the guidelines of the Association for Assessment and Accreditation of Laboratory Animal Care International. MV4-11 cells (5×10⁶ cells per mouse) were suspended with 50% Matrigel (Becton Dickinson) and subcutaneously implanted into both right and left flanks of the athymic nu/nu mice. When tumors were grown between 100 to 200 mm³, treatments were initiated. Mice were randomly assigned into two cohorts for the anti-tumor growth activity studies, with 6 mice per group. Curcumin was given intraperitoneally as a solution of a mixture of DMSO: ethanol:saline (10:3:7) at the dose of 100 mg/kg five days a week, and the placebo formulation was used as a control for 4 weeks. Tumor volume was calculated by using the equation V=2×A×B²/3, where A is the longer (horizontal as width) diameter (mm) and B is the shorter (perpendicular as depth) diameter (mm) and expressed in cubic millimeter. After 4 weeks, mice were euthanized and tumor tissues were excised, snapfrozen in liquid nitrogen, weighed and stored in a −80° C. freezer until analysis.

### Results

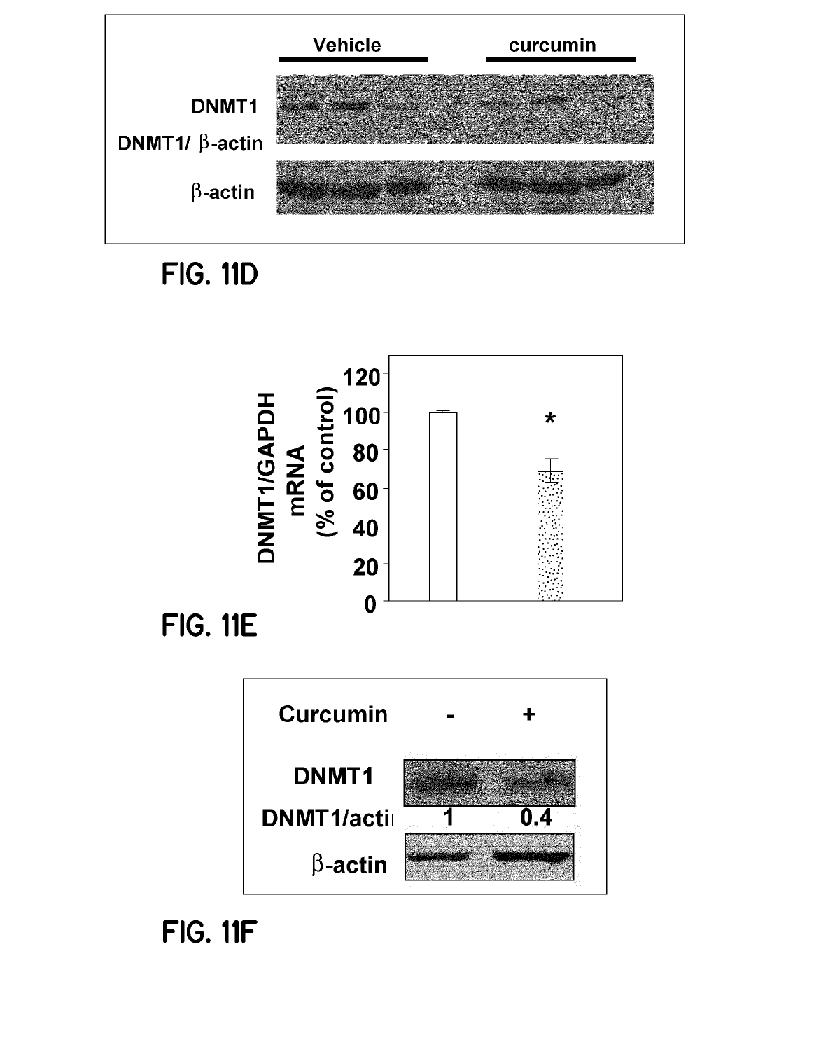

Down-regulation of DNMT1, DNMT3a and DNMT3b in MV4-11 cells in vitro and in vivo and in the primary leukemia cells from a patient with AML. Having observed disruption of all three DNA protein (Sp1) complexes on DNMT1 promoter regions in curcumin-treated MV4-11 cells in time and concentration-dependent manner, we evaluated whether the transcript level of DNMT1 was altered in curcumin-treated MV4-11 cells. As shown in the top panel in FIG. 11A, there is 70% and 90% decrease in the level of DNMT1 transcripts in MV4-11 cells treated with 10 nM curcumin for 48 hours and 72 hours compared to the untreated control, respectively.

DNMT1 is a maintenance enzyme for established DNA methylation pattern in mammary cells, whereas DNMT3a and 3b are two DNMTs responsible for the de novo methylation pattern of mammary cells. A recent study demonstrated the presence of several Sp-1 binding sites in the upstream of the DNMT3a and DNMT3b transcription initiation sites. The potential involvement of these sites in DNMT3a/b regulation was demonstrated in the following aspects: (1) a GC-rich DNA-binding protein (Sp1) inhibitor mithramycin A decreases the promoter activity and mRNA expression levels of DNMT3a and DNMT3b; (2) Over-expression of Sp1 up-regulated the promoter activities of DNMT3a and DNMT3b; and (3) The physical binding of Sp1 to DNMT3a and DNMT3b promoters was confirmed by a gel shift assay. We next evaluated whether curcumin may also down-regulate DNMT3a and DNMT3b gene expression by disruption of Sp1-DNA complex. We observed that the mRNA levels of DNMT3a were 50% and 20% of the control after incubation with 10 μM curcumin for 48 and 72 h, respectively, whereas DNMT3b mRNA levels were 40% and 10%, respectively. The level of DNMT down-regulation was the least in 3 μM curcumin-treated cells, suggesting that this effect was dose-dependent. Furthermore, curcumin-induced down-regulation of DNMTs proteins was confirmed by western blot analysis. As shown in FIG. 11B, DNMT1 protein level was completely depleted in 10 μM curcumin-treated MV4-11 cells and decreased by 50% in ML-1 cells (data not shown). The protein levels of DNMT3a and 3b were also reduced by 50% in MV4-11 cells, which was similar to that of decitabine. These results demonstrated that curcumin down-regulates DNMTs in both mRNA and protein levels in MV4-11 cells in a dose- and time-dependent manner. However, reduction in DNMTs protein expression in decitabine-treated MV4-11 cells seems to be independent on their transcription, which is consistent with its known molecular mechanism, by which decitabine traps DNMT1 covalently via its incorporation into DNA. We further extended our studies in MV4-11 cells-engrafted animal tumor model. Curcumin treatment significantly decreases the mRNA level of DNMT1 and DNMT3a, but not DNMT3b (FIG. 11C), and the protein levels of DNMT1 ((FIG. 11D, 50% decrease, p=0.044%, two way ANOVA Analysis, n=6) in the engrafted tissues, when compared with the untreated controls. More importantly, we also showed in our ex vivo study that curcumin can down-regulate DNMT1 mRNA (FIG. 11E) and protein level (FIG. 11F) in AML patient primary blast cells.

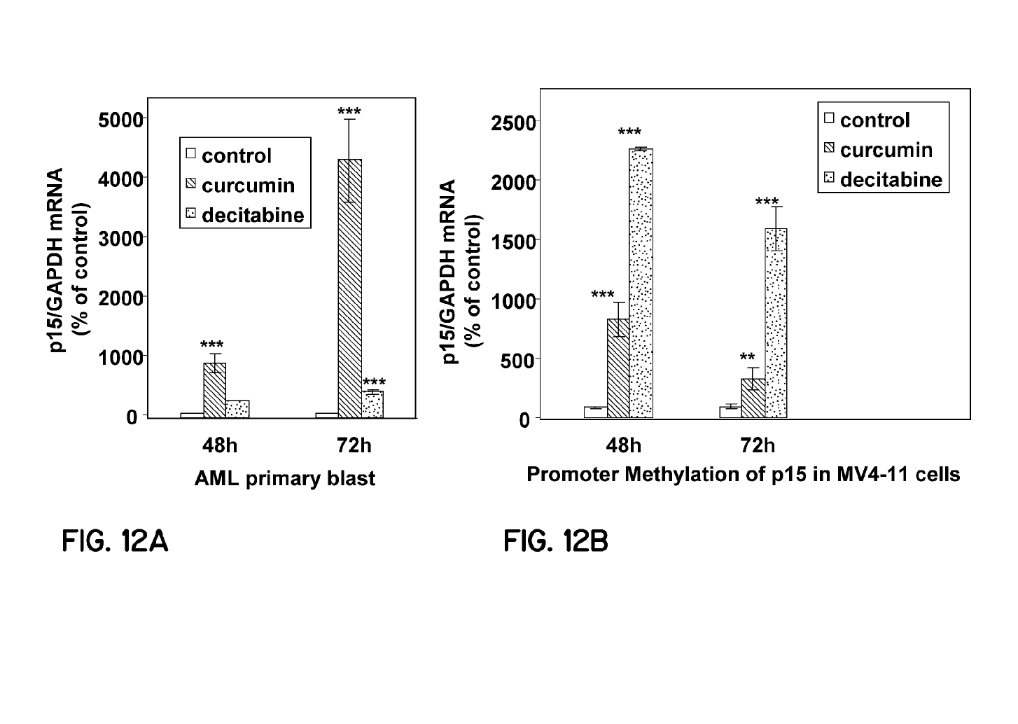

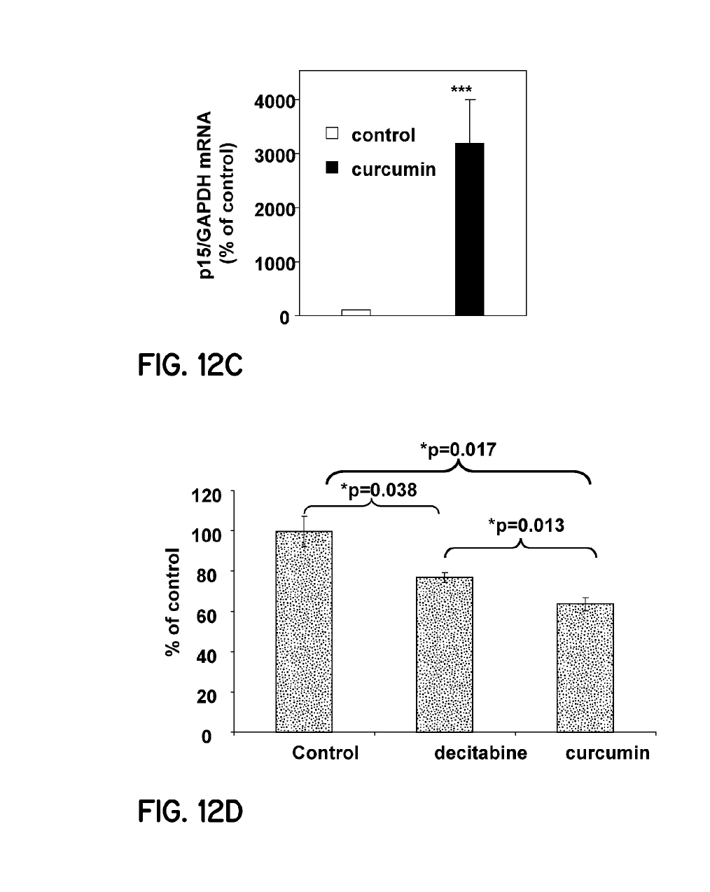

Hypomethylation is associated with the reactivation of epigenetically silenced cell-cycle-regulating TSG p15^{INK4B} in MV4-11 cells treated with curcumin. Since curcumin down-regulates DNMTs and induces global DNA hypomethylation, a logical question to ask is whether curcumin can reactivate epigenetically silenced TSGs in leukemia cell lines. p15^{INK4B} is silenced, at least partially, by its promoter hypermethylation in MV4-11 cells, and in primary blasts obtained from patients with MDS and AML at high frequency. p15^{INK4B} hypermethylation is associated with poor prognosis in MDS and AML. It has been shown that p15^{INK4B} can be reactivated in cancer cells by miR29b in the preclinical setting. These suggest that reactivation of p15^{INK4B} may serve as an effective pharmacodynamic endpoint to monitor the therapeutic effect of DNA methylation inhibitors in AML and MDS. Therefore, we evaluated the expression level of p15^{INK4B} in curcumin-treated MV4-11 and HL-60 cells. As expected, the mRNA level of p15^{INK4B} was dramatically increased following curcumin treatment in both cell lines. As shown in FIG. 12A, the expression level of p15^{INK4B} in MV4-11 cells was increased by approximately 12 and 43 fold following treatment with 10 μM curcumin at 48 hours and 72 hours, respectively. Similarly, p15^{INK4B} was also reactivated in HL-60 leukemia cells by curcumin in a time-dependent manner (FIG. 12B). More importantly, p15^{INK4B} was also reactivated by curcumin in AML primary blast cells (FIG. 12C). To evaluate whether reactivation of p15^{INK4B} is associated with its promoter hypomethylation, the methylation levels of the promoter region of p15^{INK4B} in MV4-11 cells treated with 10 μM curcumin and decitabine (2.5 μM) for 72 hours were evaluated using a recently published LC-MS/MS method. As shown in FIG. 12D, the promoter methylation level decreases about 40% and 25% in curcumin-(p=0.017, n=3) and decitabine- (p=0.038, n=3) treated MV4-11 cells, respectively, which was consistent with their enhanced expression levels induced by curcumin (˜43 fold) and decitabine (˜4 fold) (FIG. 12A).

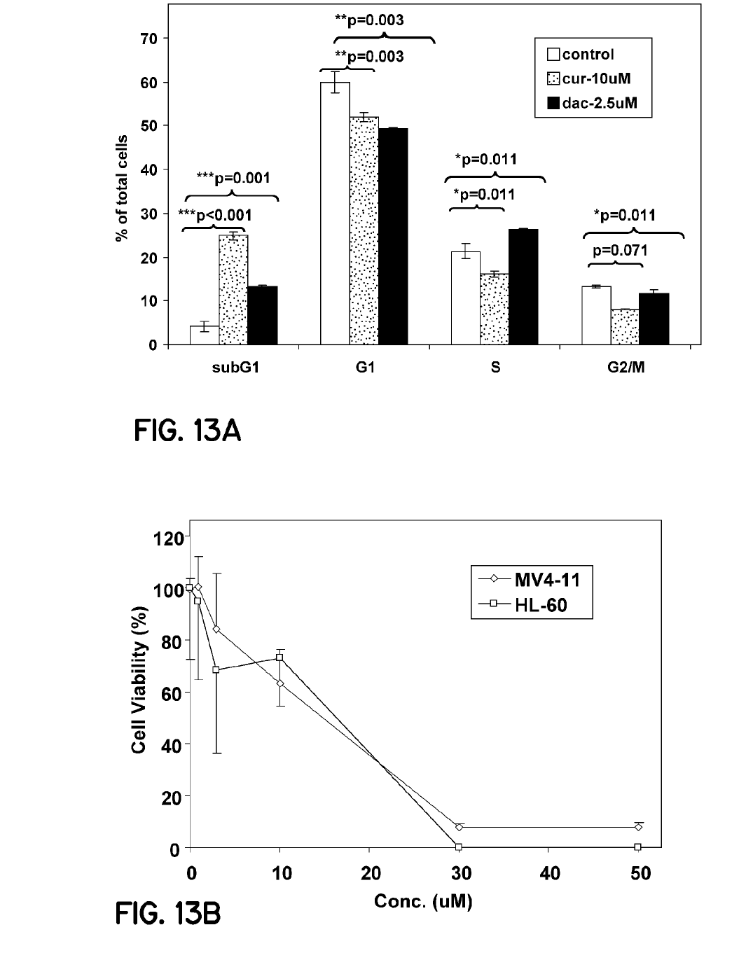

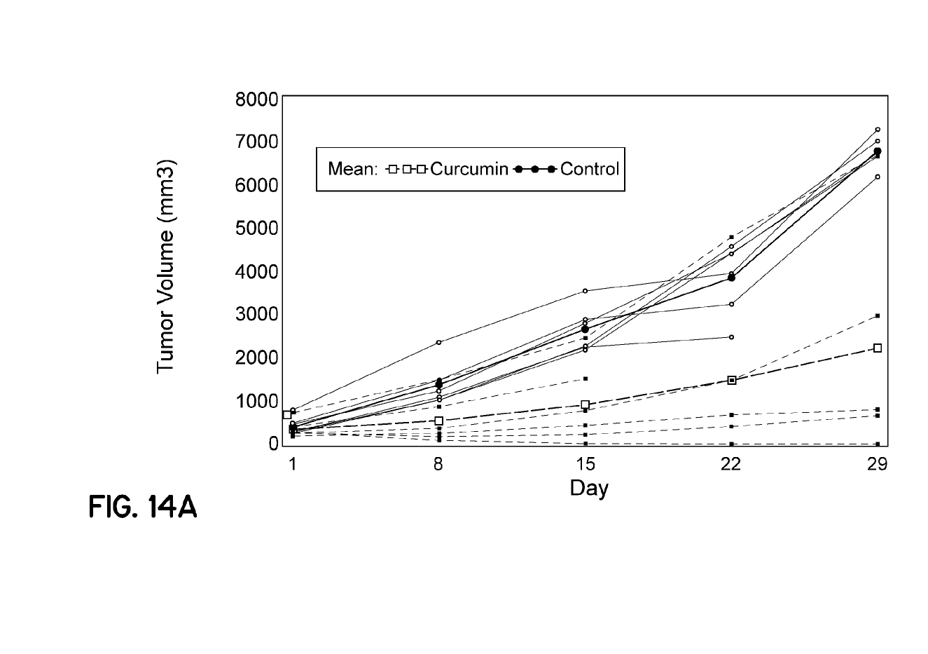

Curcumin arrests cell cycle in SubG1 phase and induces apoptosis and their associated anti-proliferation activity in MV4-11 cells. Next, the cell cycle distribution of MV4-11 cells was analyzed by flow cytometry after a 48 hour treatment with curcumin. As shown in FIG. 13A, more than half of untreated MV4-11 cells (60%) were in the G1 phase and about 4.2%, 21.2%, and 12.3% of cells were in the SubG1, S and G2/M phase, respectively, while there was significant change of the cell population distribution of SubG1, G0/G1, S and G2/M phase with values of 24.9, 52.0%, 16.1 and 8.0% in 10 μM curcumin-treated MV4-11 cells and 13.2%, 49.4%, 26.2%, and 11.7% in 2.5 μM decitabine-treated MV4-11 cells, respectively. This data suggested that curcumin arrests MV4-11 cells in the SubG1 phase and decreases the S-phase population at 10 μM, which is consistent with increase expression level of p15^{INK4B} in 10 μM curcumin-treated MV4-11 cells. In contrast, decitabine increased the S-phase cell population from 21.2% to 26.2% (*p=0.011, two sample t-test, n=4 for control and n-3 for decitabine, FIG. 13A), which is consistent with its known S-phase arrest and S-phase-dependent hypomethylation activity.

In addition to its cell cycle arrest, a significant portion of apoptotic cells (˜30%) was observed in 10 μM curcumin-treated MV4-11 cells when compared with the untreated control (˜7%), which may associate with its inhibition of apoptotic-induced transcription factor NF-KB nucleus translocation and its down-stream apoptotic genes e.g. down-regulation of Bcl-2 (data not shown).

Since curcumin can induce cell cycle arrest and apoptosis in MV4-11 cells, it may have cytotoxic activity in AML cells. As shown in FIG. 13B, a concentration-dependent cytotoxic activity was observed in both MV4-11 and HL-60 cells, the cell viability was decreased to 60-70% in these two cell lines, respectively. Similar cytotoxicity of curcumin was also observed in K562 with an IC₅₀ of 10.4 μM, respectively.

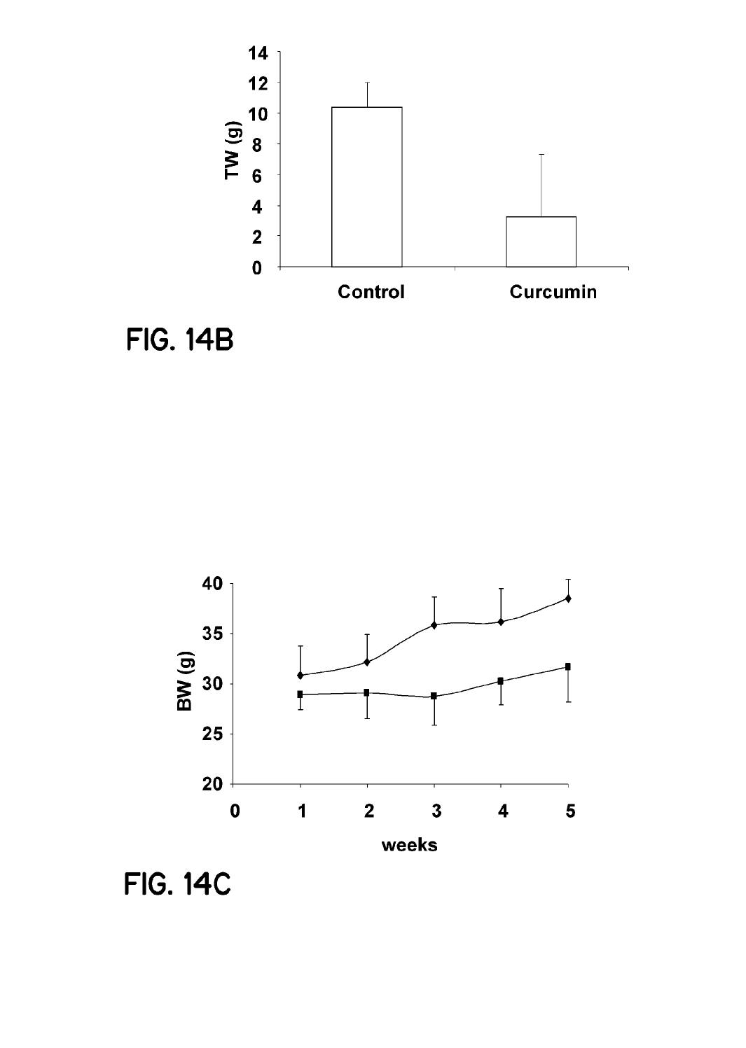

The curcumin formulations according to embodiments of the invention exhibit in-vivo anti-tumor growth activity. The in-vivo anti-tumor growth activity of curcumin was further evaluated in a MV4-11 leukemia cell-engrafted animal model. As shown in FIG. 14A, significant anti-tumor growth activity of curcumin as the inhibitive growth rate of the tumor. A linear mixed effect model analysis demonstrated that compared with control, curcumin decreased tumor growth rate by 142.5 mm³ per day (p-value <0.0001). The decrease in the tumor growth rate as the slopes is corresponding to about 70% inhibition of tumor growth in nude mice treated with i.p. administration of 100 mg/kg to mice. We also compared the tumor sizes and weights in the two groups at day 29 after the treatment, it revealed that curcumin significantly reduced the tumor size by 3854 mm³ (p-value <0.0001) and weight by 65% (p-value=0.002) (FIG. 14B).

No significant weight loss and a slight weight gain have been documented for both placebo and curcumin-treated group (FIG. 14B). The slight weight gain is probably due to the tumor growth, as the average tumor weight for the placebo group was 10.4 g and for the treatment group was 3.6 g (FIG. 14C). Additionally, a pilot study demonstrated that curcumin significantly increased the life span of MV4-11 engrafted nude mice from 4 weeks in the placebo group to 6 weeks in curcumin-treated group (data not shown).

Curcumin has potent inhibitory activity on CpG methyltransferase (M. SssI) and induces significant global DNA methylation in an AML MV4-11 cell line. DNA methylation patterns are regulated and maintained by several methyltransferases in mammary cells, including DNMT1, DNMT3a and DNMT3b. These data presented herein demonstrated that curcumin decreases the mRNA and protein level of DNMT1, DNMT3a and DNMT3b via their transcription modulation effect. Down-regulation of DNMT1 may be associated with either disruption of NF-κB/DNA or Sp1/DNA complex binding to the promoter region of DNMT1 or cell cycle SubG1 arrest. The dual functions of curcumin as chemical inhibitory effect and transcript modulation of DNMTs result in its hypomethylation activity as the marginal global DNA hypomethylation. The discovery that curcumin downgulates not only DNMT1 but also DNMT3a and DNMT3b has important functional ramifications, since it has been reported that selective genetic disruption of DNMT3b in colon cancer cell lines reduced GDM only by 3% and selective genetic disruption of DNMT1 in colon cancer cell lines reduced GDM by 20%. However, genetic disruption of both DNMT1 and DNMT3b, completely abolishes DNA methyltransferase activity and reduced GDM by 95%. Consistent with these results, the data herein demonstrate that curcumin can efficiently modulate DNA hypomethylation because of its targeting of both DNMT3s and DNMT1. Curcumin in the formulations prepared according to embodiments of the invention, reactivate a promoter-hypermethylation silenced tumor suppressor gene p15^{INK4B} in a dose- and time-dependent manner and the reactivation is at least partially associated with the promoter hypomethylation of p15^{INK4B}. Importantly, reactivation of p15^{INK4B} along with reactivation of p21, possibly associated with its modulation effect on histone acetylation (data not shown), may be associated with its anti-cancer activities, e.g. anti-proliferative and cell cycle arrest. The data demonstrate the anti-proliferation activity of the present curcumin formulations in several AML cell lines, e.g., MV4-11 and HL-60 in the range of its IC₅₀ of 10-15 μM (FIG. 13B) and sub-G1 arrest of MV4-11 cells by curcumin. Consistent with its in-vitro anti-proliferation and cell-cycle arrest activity, a significant anti-tumor growth activity was also documented in MV4-11 engrafted nude mice with about 65% tumor size decrease and 70% tumor growth rate. Additionally, the survival life span of curcumin-treated MV4-11 engrafted nude mice was found to be at least one week longer than that of the placebo-treated mice. Taken together, the present curcumin formulations are effective DNA methylation inhibitors and have significant anti-tumor growth activity on AML cell lines in-vitro and in-vivo.

Importantly, the hypomethylation activity of curcumin appears to be S-phase independent, which is different from that of azanucleosides as S-phase specific, requiring incorporation into DNA. Therefore, the present curcumin formulations have following advantages over azanucleoside hypomethylation agents: (1) curcumin exerts hypomethylation activity without entailing the majority of tumor cells passing through S-phase and preceded via replication; (2) curcumin exerts hypomethylation activities on non-replicating subpopulations of cells and subpopulations of cells with stem cell like properties exist, which are generally non-replicating and may be particularly difficult populations of cells to treat with azanucleoside hypomethylation agents.

Additionally, it is expected that the response of patients to azanucleoside hypomethylation agents is associated with several key emzymes e.g. cytidine deaminase and kinase, which deactivates azanucleosides by replacement of their 4-amino group to their corresponding deaminated metabolites (4-OH) or activate azanucleosides by phosphorylation to their corresponding triphosphates, a precursor to be incorporated into DNA, and eventually to capture DNMT1 resulting in their hypomethylation activities. For example, the high expression of cytidine deaminase in patients could limit their anti-tumor effectiveness and develop tumor resistance to azanucleoside hypomethylation agents. In spite of no data on the effect of curcumin on cytidine deaminase, it is expected that curcumin would not be the substrate of cytidine deaminase, therefore curcumin may provide an complementary hypomethylation agent in these resistant tumor cells to azanucleosides.

Another pitfall for azanucleoside hypomethylation agents is that it remains unknown what effect prolonged hypomethylation and epigenetic activation has on other non-cancer related genes, especially in the normal tissues. Some investigators have raised the concern that the longer term use of hypomethylation agents could itself be tumorogenic although both chemopreventive and tumor promoter effects of reduced DNMT expression have been observed in mice. However, in the case of curcumin, it would not be a big concern for its hypomethylation activity associated tumorogenicity since curcumin has been long-time used in human and well tolerated up to 8 g/day for 3-4 months without documentation of any significant toxicities. What's more, comprehensive and extensive significant healthy benefit of curcumin has been documented for various human chronicle diseases including aging and cancers.

Based on the difference in mechanisms of activity and potential advantages and complementarities of curcumin over azanucleosides, it would not be unreasonable to evaluate possible synergism by using the combination of curcumin and azanucleosides in hematologic malignancies. Such a combination is not expected to increase the myelotoxicity, a dose limiting toxicity associated with the use of azanucleosides (e.g., acute leukemia and high-risk MDS), since curcumin is considered a non-toxic and well-tolerated dietary supplement.

### Example 4

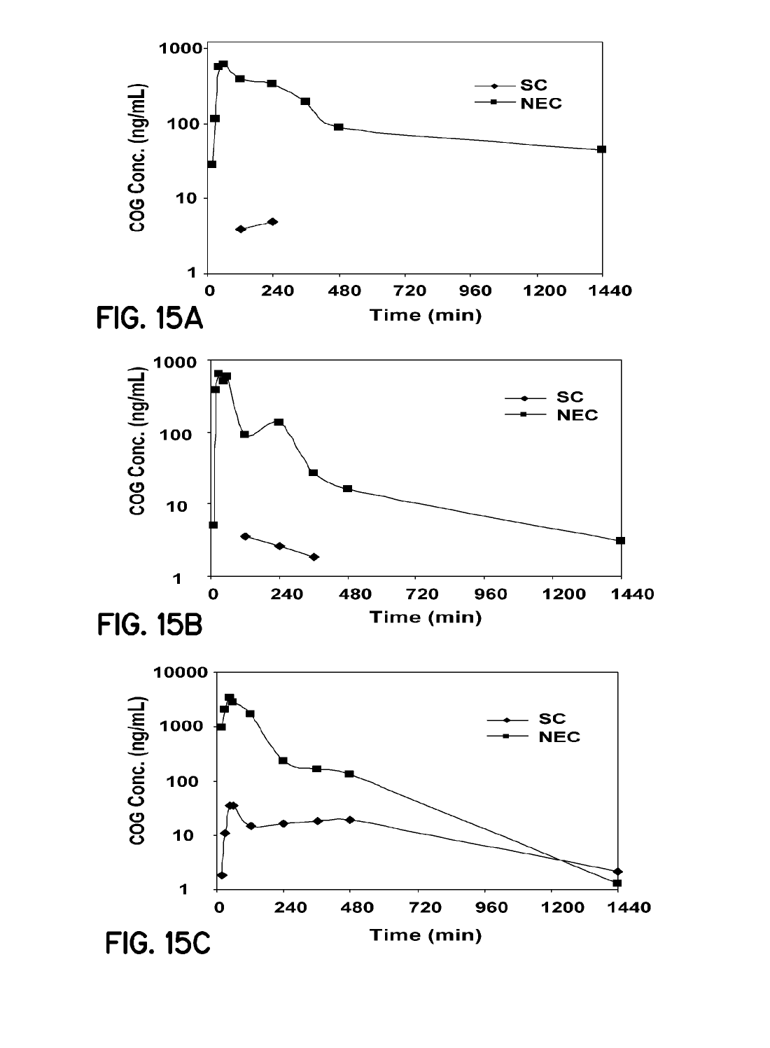

The curcumin formulations according to embodiments of the invention significantly increase oral absorption of curcumin in humans. We characterized the plasma concentration of curcumin and COG in four healthy volunteers that were administered a suspension of curcumin and in three subjects administered the emulsified curcumin formulations prepared according to embodiments of the invention (“emulsified curcumin”) We were unable to detect plasma curcumin levels in all subjects after oral administration of suspended curcumin and in one subject after oral administration of emulsified curcumin Detectable plasma levels of curcumin ranged between 3-8 ng/mL (3-8 ng/mL) following administration of emulsified curcumin in two subjects. The major metabolite, curcumin-O-glucoronide (COG), was detected in all subjects. Notably, significantly higher peak plasma levels (Cₘₐₓ) of COG (200 to 350 fold higher) were detected with emulsified curcumin compared to the curcumin suspensions as early as 10 min and up to 24 hr as shown in the individual composite plasma concentration time profile of COG in three subjects as shown in FIG. 15A to 15C. The AUC₀₋₂₄ hr of COG is about 80 to 600 fold higher for emulsified curcumin compared to the suspension of curcumin Non-compartment PK analysis demonstrated that terminal elimination half lives of COG is about 6 hr after emulsified curcumin administration, which is similar after the curcumin suspension administration as shown in one female subject (FIG. 15C). Additionally, the urine level of curcumin and COG was measured in several early time points in one subject. It was found that (1) both curcumin and COG can be detected in human urine; (2) the urinary peak concentrations of curcumin and COG were 17.1 and 12.9 ng/mL, and 1470 and 256 ng/mL at 2 hr following an oral administration of emulsified curcumin and the curcumin suspension, respectively; (3) 3-fold and 12-fold higher urinary levels of curcumin and COG were observed with the emulsified curcumin compared with the curcumin suspension, respectively. When we take the data collectively, we propose that the plasma AUC of COG and ratio of COG: curcumin should be better indicators of curcumin absorption. In summary, (1) this is the first report of both curcumin and COG in plasma and urine after an oral administration of emulsified curcumin and curcumin C3 complex in human; (2) non-detectable to low plasma levels up to 124 ng/mL (mean: 33+DS ng/mL) of COG at 2 hr in previous clinical studies suggest that the indirect method for Phase II metabolites quantification may underestimate the actual concentrations. Therefore, our method provides a unique and accurate method for simultaneous quantification of curcumin and COG without potential bias introduced by enzymatic hydrolysis; and, (3) comparative analysis of curcumin and COG in plasma and urine suggest that the urine concentrations of curcumin is higher and the time to reach the peak level of curcumin in urine is longer than those in plasma, which may implicate that COG can convert to curcumin after COG enters the circulating system.

What we claim is:

1. A pharmaceutical composition comprising:
curcumin, and
at least two excipient polymers selected from the group consisting of a polyethoxylated castor oil, a polyoxyethylene sorbitan ester, and a polyethylene glycol (PEG).

2. The composition of claim 1, wherein the at least two excipient polymers are a polyethylene glycol and at least one of a polyethoxylated castor oil and a polyoxyethylene sorbitan ester.

3. The composition of claim 1, wherein the polyethylene glycol has an average molecular weight selected from the group consisting of 200, 300, 400, 500, 600, and combinations thereof, and wherein the polyoxyethylene sorbitan ester is selected from polysorbate 20, 30, 40, 50, 60, 65, 70, 80, 85, and combinations thereof.

4. The composition of claim 1, wherein the polyethoxylated castor oil is the reaction product of 35 moles of ethylene oxide with each mole of castor oil.

5. The composition of claim 1, wherein a volume ratio of a polyethylene glycol to a second excipient polymer ranges from about 1:99 to about 99:1.

6. The composition of claim 5, wherein the second excipient polymer is the polyethoxylated castor oil and the volume ratio is about 4:1 to about 1:1.

7. The composition of claim 5, wherein the second excipient polymer is the polyoxyethylene sorbitan ester and the volume ratio is about 10:1 to about 1:1.

8. The composition of claim 1, wherein the composition is a gel having a curcumin concentration within the range of about 40 mg/mL to about 250 mg/mL.

9. The composition of claim 7, wherein the curcumin concentration is within the range from about 100 mg/mL to about 200 mg/mL.

10. A method of making an oral gel pharmaceutical composition comprising
forming a curcumin suspension in a liquid comprising a first excipient polymer selected from the group consisting of a polyethoxylated castor oil, a polyoxyethylene sorbitan ester, and a polyethylene glycol (PEG);
heating the curcumin suspension to a temperature between a range from about 30° C. to about 150° C. to form a homogenous curcumin gel with a curcumin concentration within a range of about 50 mg/ml to about 250 mg/ml; and
diluting the homogenous curcumin gel with a liquid comprising a second excipient polymer selected from the group consisting of a polyethoxylated castor oil, a polyoxyethylene sorbitan monolaurate, and a polyethylene glycol (PEG).

11. The method of claim 10, wherein the curcumin concentration of the homogenous curcumin gel is within the range from about 130 mg/mL to about 250 mg/mL.

12. A method of increasing a biovailability of curcumin, the method comprising:
administering an oral gel pharmaceutical composition to a subject, wherein the pharmaceutical composition comprises:
curcumin, and
at least two excipient polymers selected from the group consisting of a polyethoxylated castor oil, a polyoxyethylene sorbitan monolaurate, and a polyethylene glycol (PEG).

13. The method of claim 12, wherein the at least two excipient polymers are a polyethylene glycol and at least one of a polyethoxylated castor oil and a polyoxyethylene sorbitan ester.

14. The method of claim 12, wherein the polyethylene glycol has an average molecular weight selected from the group consisting of 200, 300, 400, 500, 600, and combinations thereof, and wherein the polyoxyethylene sorbitan ester is selected from polysorbate 20, 30, 40, 50, 60, 65, 70, 80, 85, and combinations thereof.

15. The method of claim 12, wherein the polyethoxylated castor oil is the reaction product of 35 moles of ethylene oxide with each mole of castor oil.

16. The method of claim 12, wherein a volume ratio of a polyethylene glycol to a second excipient polymer ranges from about 1:99 to about 99:1.

17. The method of claim 16, wherein the second excipient polymer is the polyethoxylated castor oil and the volume ratio is about 4:1 to about 1:1.

18. The method of claim 16, wherein the second excipient polymer is the polyoxyethylene sorbitan ester and the volume ratio is about 10:1 to about 1:1.

19. The method of claim 12, wherein a curcumin concentration in the composition is within the range of about 40 mg/ml to about 250 mg/ml.

20. The method of claim 19, wherein the curcumin concentration is within the range from about 100 mg/ml to about 200 mg/mL.

21. A method of treating a disease sensitive to curcumin comprising:
administering to a subject in need of such treatment a therapeutically effective amount of a pharmaceutical composition comprising:
curcumin, and
at least two excipient polymers selected from the group consisting of a polyethoxylated castor oil, a polyoxyethylene sorbitan ester, and a polyethylene glycol (PEG),
wherein the disease is characterized by abnormally rapid proliferation of tissue involved in said disease which is mediated by or associated with abnormally increased levels of DNA methylation, and wherein the therapeutically-effective amount of the pharmaceutical composition is sufficient to modulate DNA methylation.

22. The method of claim 21, wherein the therapeutically-effective amount is sufficient to inhibit DNA methylation.

23. The method of claim 21, wherein said subject is human.

24. The method of claim 21, wherein said subject is a domesticated animal.

25. A method for treating cancer in a subject comprising:
(a) administering to the patient a therapeutically-effective amount of a pharmaceutical composition comprising:
curcumin, and
at least two excipient polymers selected from the group consisting of a polyethoxylated castor oil, a polyoxyethylene sorbitan ester, and a polyethylene glycol (PEG).

26. The method of claim 25, wherein said subject is human.

27. The method of claim 25, wherein said subject is a domesticated animal.

28. The method of claim 25, wherein said cancer is leukemia or breast cancer.
